(19) United States
(12) Patent Application Publication
(10) Pub. No.: US 2025/0161686 A1
(43) Pub. Date: May 22, 2025

(54) MULTI-SITE NEUROMODULATION FOR TREATMENT OF ALS AND RELATED DISORDERS

(71) Applicant: Research Foundation of the City University of New York, New York, NY (US)

(72) Inventor: Zaghloul Ahmed, Staten Island, NY (US)

(21) Appl. No.: 19/035,454

(22) Filed: Jan. 23, 2025

Related U.S. Application Data
(63) Continuation-in-part of application No. 17/331,142, filed on May 26, 2021, now Pat. No. 12,208,266.
(60) Provisional application No. 63/187,745, filed on May 12, 2021.
(60) Provisional application No. 63/187,116, filed on May 11, 2021.
(60) Provisional application No. 63/029,816, filed on May 26, 2020.
(60) Provisional application No. 63/647,442, filed on May 14, 2024.

(51) Int. Cl. A61N 1/36; A61N 1/05

(52) U.S. Cl. CPC A61N 1/36121; A61N 1/0529; A61N 1/36082

(57) ABSTRACT

Methods and systems for treating ALS and related motor neuron diseases using multi-site neuromodulation are disclosed.

### CROSS-REFERENCE TO RELATED APPLICATIONS

This application claims priority to, and is a continuation-in-part of, U.S. non-provisional application Ser. No. 17/331,142 (filed May 26, 2021) which is a non-provisional of U.S. provisional patent applications 63/029,816 (filed May 26, 2020); 63/187,116 (filed May 11, 2021) and 63/187,745 (filed May 12, 2021), the content of which are incorporated herein by reference for all purposes.

### FIELD

The present invention relates to methods and systems based on multi-site neuromodulation for treating amyotrophic lateral sclerosis (ALS) and related neurological disorders.

### BACKGROUND

Amyotrophic lateral sclerosis (ALS, Lou Gehrig's disease, Charcot's disease) is a rapidly fatal progressive neurodegenerative disease that affects multiple neuronal networks. The disease is the result of a systematic dismantling of the motor neuron system, with clinical manifestations dependent on site of onset; the relative affinity of the dismantling process for prefrontal, upper and lower motor neurons; and the rate of the disease's spread within the network (Ravits et al., “ALS motor phenotype heterogeneity, focality, and spread: deconstructing motor neuron degeneration”, Neurology, 2009). ALS results in muscle weakness, spasticity, paralysis and eventual death, typically within 2-5 years of diagnosis. Mechanism-specific treatments directed at the processes that cause ALS to evolve after it has expressed itself sufficiently to be diagnosed may, at best, have an ameliorative effect. Treatments that halt the spread of the disease may be more effective than those that try to salvage affected motor neurons, though such treatments have yet to be realized. Currently, ALS therapy consists of a few disease-modifying treatments directed at specific pathophysiological mechanisms implicated in ALS. There are at present two FDA-approved small molecule drugs and one FDA-approved anti-sense oligo (ASO) for the treatment of ALS, all having limited impact on disease progression. There is tremendous unmet medical need for entirely new approaches to treating ALS, especially modalities based on non-invasive neuromodulation.

While the majority of cases (90-95%) of ALS are sporadic and occur in patients without known familial history of ALS, the remaining 5-10% of cases are familial. There are at least 31 different gene mutations that have been associated with familial ALS (Mathis et al., “Genetics of amyotrophic lateral sclerosis: a review”, J. Neurol. Sci., 2019), and the most common types of familial ALS are shown in Table 1:

TABLE 1
Most common subtypes of
familial ALS by gene mutation

| | % of | |
|---|---|---|
| Mutation | familial ALS | Predominant dysfunction (7) |
| C9orf72 | 30% | Both UMN and LMN |
| SOD1 | 20% | Both UMN and LMN, but LMN |
| | | becomes dominant with disease |
| | | progression |
| TARDBP | 5% | Both UMN and LMN |
| FUS | 5% | Both UMN and LMN, but LMN |
| | | becomes dominant with disease |
| | | progression |

Clinical presentation in ALS includes fasciculations, muscle cramps, hyper-reflexia and spasticity, which typically progresses to shortness of breath, ventilatory dysfunction and eventual respiratory failure. ALS patients can exhibit both UMN and LMN symptoms. UMN symptoms include spasticity, weakness and increased reflexes, while LMN symptoms include muscle atrophy, weakness, fasciculations and decreased reflexes. Patients with ALS often complain of painful muscle cramps, spasms and spasticity. Patients with primary lateral sclerosis (PLS), a phenotype associated with predominant UMN impairment, can have severe spasticity (Singer et al., “Primary lateral sclerosis”, Muscle Nerve, 2007).

Recent research has established important links between ALS and motor neuron hyperexcitability. In this pathophysiological mechanism, hyperexcitable motor neurons depolarize excessively, become fatigued, and eventually die, leading to weakness and paralysis in the muscles innervated by those motor neurons. Human neurophysiological studies using nerve conduction testing have demonstrated axonal hyperexcitability of motor neurons in both sporadic and familial types of ALS, and hyperexcitability has been reported in other ALS subtypes, suggesting that motor neuron hyperexcitability is consistently found across different ALS variants. Cortical hyperexcitability has also been shown in ALS, and is an early feature of ALS which may precede muscle weakness onset.

The formation of protein aggregates in the cytoplasmic compartment of motor neurons is another central feature of ALS pathology. These aggregates typically occur in the form of ubiquitinated inclusions and can contain various proteins, including SOD1, TDP-43, FUS, OPTN and other proteins that get trapped in the inclusions. These protein aggregates have toxic effects on the motor neuron through toxic gain-of-function or toxic loss-of-function and can have prion-like properties.

As will be appreciated by a person skilled in the art, use of the terms disease, disorder, dysfunction and degradation, alone or in combination in this document shall be understood as interchangeable.

### SUMMARY OF THE INVENTION

The present invention is directed to treatment and reversal of motor neuron diseases and neurodegenerative diseases, including ALS. In embodiments of the present invention, we address the need for regulation of protein degradation pathways and autophagy and the need for decreasing cytosolic aggregation of TDP-43, and similar proteins that aggregate in the cytoplasmic compartment of neurons.

We describe herein that NKCC1 is elevated in SOD1-G93A mice, and that stimulation with our non-invasive multi-site direct current stimulation (abbreviated “msDCS”) neuromodulation technology results in reduction of elevated NKCC1 levels, thereby reducing the hyperexcitability of the neurons by normalizing chloride gradient. We further describe herein additional biochemical changes in spinal motor neurons following stimulation, as well as findings that stimulation results in reduction of tremor, slowing of disease progression, improvement of motor function, increased preservation of spinal motor neurons and increased survival in SOD1-G93A mice. We also describe herein biochemical changes in spinal motor neurons following stimulation in the TDP-43 mouse model of ALS, as well as findings that stimulation results in improvement of motor function in such TDP-43 mice.

There are numerous proteins that are known to form pathological aggregates in the cytoplasmic compartment of neurons during the course of ALS and other neurodegenerative diseases. The gene product of the TARDBP gene listed in Table 1 is a protein known as TAR DNA binding protein 43 (TDP-43), and is one example of a protein known to aggregate in the cytoplasm of motor neurons in ALS patients. TDP-43 has functions which include the regulation of RNA metabolism, mRNA transport, microRNA maturation and stress granule formation. Aggregated cytoplasmic TDP-43 protein is found in motor neurons of 97% of ALS cases (sporadic and familial). TDP-43 inclusions are also found in many other neurodegenerative diseases, shown in Table 2, indicating that this protein plays a common role in neurodegeneration.

TABLE 2
Neurodegenerative diseases linked to TDP-43 pathology

| | | Co- | | | |
|---|---|---|---|---|---|
| Disease associated | | occurence | Estimated # | | |
| with TDP-43 | Predominant | of TDP-43 | of US | | |
| proteinopathy | pathology | pathology | patients | | Comments |
| Classic ALS | TDP-43 | | ~30,000 | | TDP-43 aggregation found |
| | | | | | in 97% of ALS cases |
| Familial SOD1 ALS | SOD1 | TDP-43 | ~1,000 | | SOD1 aggregation found |
| | | rare | | | in 2-5% of ALS cases |
| Progressive muscular | TDP-43 | | ~1,000-2,000 | | Most frequent is TDP-43 in |
| atrophy (PMA) | | | | | UMN/LMN. Also can be |
| | | | | | LMN only |
| ALS-FTLD | TDP-43 | | ~4,000-5,000 | | FTLD found in about 15% |
| | | | | | of ALS cases |
| FTLD | TDP-43 | | ~60,000-70,000 | | FTLD (TDP-43) accounts |
| | | | | | for 50% of FTLD cases |
| Alzheimer's | β-amyloid, tau | TDP-43 | ~6.7 | million | TDP-43 aggregates found |
| disease (AD) | | | | | in 57% of AD brains |
| Dementia with | α-synuclein | TDP-43 | ~1.4 | million | |
| Lewy bodies | | | | | |
| Primary lateral | TDP-43 | | ~500-2,000 | | TDP-43 inclusions in |
| sclerosis (PLS) | | | | | frontotemp. cortex, |
| | | | | | subcortical grey |
| | | | | | matter |
| Parkinson's disease | α-synuclein | TDP-43 | ~1 | million | |
| Huntington's disease | Huntingtin | TDP-43 | ~30,000 | | |
| | protein | | | | |
| Hippocampal | TDP-43 | | Data not | | TDP-43 inclusions in |
| sclerosis | | | widely | | hippocampus with |
| | | | available | | neuronal loss |
| LATE/CARTS | TDP-43 | | ~1 | million | TDP-43 in limbic |
| | | | | | system, hippocampus |
| | | | | | and other regions |
| Chronic traumatic | Tau | TDP-43 | Thousands | | TDP-43 found in |
| encephalopathy | | | | | nearly all cases of |
| | | | | | CTE |
| Perry disease | TDP-43 | | <50 | cases WW | TDP-43 found in |

| | | | |
|---|---|---|---|
| | | | extrapyramidal |
| | | | system and brain |
| | | | stem |
| Multisystem | TDP-43 | rare, familial | TDP-43 found in |
| proteinopathy | | | muscle |

Multiple processes have been implicated in TDP-43 pathogenesis, including the following pathologies: 1) nuclear depletion of TDP-43, which is associated with excessive accumulation of the protein in the cytoplasm; 2) cytoplasmic aggregation of hyperphosphorylated TDP-43 that could cause cellular stress, aberrant stress granule formation, mitochondrial dysfunction, reduced autophagy, and dysfunction of proteasomal processes; and 3) reduced expression of heat shock proteins (HSPs). TDP-43 has prion-like behavior which might explain its involvement in the anatomical progression of ALS. The two main cellular processes that work to degrade and clear TDP-43 aggregates are the proteasomal degradation and autophagy pathways, and failed TDP-43 clearance through these pathways has been implicated in ALS. As toxic effects of TDP-43 are dose-dependent, an intervention with the present invention can reduce the presence of cytosolic TDP-43 aggregates to rescue degenerative processes in motor neurons and bring the motor neurons back to a less dysfunctional and healthier state.

In an illustrative practice of the presently disclosed msDCS neuromodulation invention, we activate protein degradation pathways using our non-invasive approach, resulting in robust reduction of SOD1 and TDP-43 protein levels at and around the site of stimulation in the spinal cord. Our direct current stimulation technology described herein enhances these two protein degradation pathways in the SOD1-G93A ALS mouse model herein described, as well as a spinal cord injury model, an Alzheimer mouse model, and in vitro.

As there currently are not any FDA-approved therapeutics that dramatically increase survival in ALS, it is significant that our disclosed invention including our msDCS technology can provide an entirely new approach to suppressing the degeneration of motor neurons in ALS and other neurodegenerative diseases by simultaneously suppressing motor neuron hyperexcitability and activating protein degradation pathways.

In one or more embodiments, the present invention is directed to methods, devices and systems for treating amyotrophic lateral sclerosis and related disorders by applying a source of direct current (DC) to multiple locations along a defined neural axis in animals, including humans and other sentient beings. The locations for application of direct current include the spinal column, peripheral nerves and the cranium.

Our recent research has established important links between ALS and motor neuron hyperexcitability. We have developed a novel non-invasive approach that uses msDCS as a form of non-invasive neuromodulation, to suppress hyperexcitable spinal motor neurons.

In general, the present invention is directed to new methods, devices and systems for treatment of ALS and related neurodegenerative disorders utilizing novel applications of our msDCS system which incorporate trans-spinal direct current stimulation (tsDCS). The outcome is to suppress neuronal hyperexcitability and activate protein degradation pathways to reduce or prevent progression of the clinical manifestations of such neurodegenerative causes. In particular, we have developed a new system enabling an entirely novel approach to slowing ALS progression using non-invasive neuromodulation in a manner that suppresses motor neuron hyperexcitability while activating protein degradation pathways to reduce pathological levels of SOD1, TDP-43 and similar proteins in motor neurons.

This innovation is further applicable to treatment of related neurodegenerative diseases, including, for example, but not by way of limitation: classic amyotrophic lateral sclerosis, familial SOD1 ALS, progressive muscular atrophy (PMA), frontotemporal lobar degeneration (FTLD), ALS-FTLD, Alzheimer's disease (AD), dementia with Lewy bodies, primary lateral sclerosis (PLS), Parkinson's disease, Huntington's disease, hippocampal sclerosis, limbic-predominant age-related TDP-43 encephalopathy (LATE)/cerebral age-related TDP-43 with sclerosis (CARTS), chronic traumatic encephalopathy (CTE), and Perry disease and multisystem proteinopathy.

In SOD1-G93A mice treated non-invasively with our technology, we have shown reduction of tremor, slowing of disease progression, improvement of motor function, increased preservation of spinal motor neurons and increased survival. We have also utilized a transgenic ALS model for characterizing the effects of our msDCS. In TDP-43 mice treated non-invasively with our technology, we have shown improvement of motor function and reduction of cytosolic TDP-43 protein aggregates in spinal motor neurons in the treated mice.

We have found that NKCC1 is overexpressed, SOD1 is overexpressed, HSP70 is reduced, and phosphorylated tau is overexpressed in the SOD1-G93A model. We have found that stimulation with anodal msDCS results in suppression of NKCC1 at the intervention site. Furthermore, we found that stimulation with anodal msDCS results in a decrease in SOD1, an increase in HSP70, upregulation of LCB3 expression and a decrease in phosphorylated tau in the spinal cord. We have found that cytosolic TDP-43 protein aggregates are reduced in spinal motor neurons in TDP-43 mice.

It will be understood that the Multi-Site DCS term includes simultaneous direct current stimulation at multiple neuronal targets at different locations on the patient. This may include any combinations of spinal, cortical and peripheral neural locations. Furthermore, we include in this description a basic stimulation configuration defining a single anodal site and a single cathodal site defining a single current path for such direct current stimulation. All of these configurations are considered to be encompassed within the abbreviation “msDCS”.

Our msDCS invention is adapted to be favorably applied as a significant tool to slow ALS progression in humans. This disclosure teaches an entirely new approach to treating and slowing ALS progression. In one illustrative embodiment of the present invention, we provide non-invasive multi-site neuromodulation in a manner that suppresses motor neuron hyperexcitability and activates protein degradation pathways. In one illustrative embodiment we teach: a stimulation device for treating motor neuron diseases or neurodegenerative diseases, featuring regulation of protein degradation pathways and autophagy associated with any of the spinal cord, the brain and/or a peripheral nerve. The device further includes a direct current voltage source having a plurality of terminals; a first of the terminals for connecting a first electrode to the direct current voltage source; the first electrode being for location at or proximate to any of a dorsal aspect of a spinal cord of a vertebrate being, at or on a cranium of a vertebrate being or at or proximal to a peripheral nerve of a vertebrate being; a second of the terminals for connecting a second electrode to the direct current voltage source; the second electrode being placed at a position remote from the first electrode; the first and second electrodes being oppositely charged and configured to form a current path for stimulating the spinal cord, cranium and/or peripheral nerve; and a controller component configured to control of the current flow between the electrodes across the current path; the controller component being also configured to provide direct current flow for stimulation of of the current path across the spinal cord, cranium and/or the peripheral nerve in a manner that results in increased biological activity or gene expression or protein expression of any member of the set of biological macromolecules including HSP70, HSP72, LC3B, LC3A, or the like.

In another illustrative embodiment of the present invention we teach: a system for treatment of amyotrophic lateral sclerosis (ALS) and other neurodegenerative diseases linked to cytosolic aggregation of TDP-43 proteins in neurons of vertebrate beings. An illustrative system includes a first stimulation component configured to deliver direct current stimulation to regions of the spinal cord or brain associated with ALS or other neurodegenerative disorders linked to cytosolic aggregation of TDP-43 proteins in neurons of vertebrate beings, this stimulation component defining a therapy circuit including an active stimulation pole and a reference pole, said therapy circuit configured to provide direct current stimulation between said active stimulation pole and said reference pole; and a controller component configured to provide direct current stimulation for a predetermined time period and at a predetermined current intensity, wherein said predetermined time period and said predetermined current intensity are selected to decrease cytosolic aggregation or protein levels of TDP-43 proteins in neurons of the spinal cord or brain of vertebrate beings.

Other features and advantages of the disclosure will be apparent from the following description of some preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In addition, the materials, methods, and examples are illustrative only and not intended to be limiting.

Where applicable or not specifically disclaimed, any one of the embodiments described herein are contemplated to be able to combine with any other one or more embodiments, even though the embodiments are described under different aspects of the disclosure.

These and other embodiments are disclosed and/or encompassed by, the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, is given by way of example, and is not intended to limit the disclosure solely to the specific embodiments described, and may best be understood in conjunction with the accompanying drawings, in which:

FIG. 1A-1C show a set up for msDCS stimulation in mice. FIG. 1A describes the stimulation setup where mice have three electrodes positioned along the spinal column, two sciatic nerve electrodes, and an abdominal return electrode. The rationale behind this electrode arrangement is to drive current across the spinal cord, and down the lower limbs. FIG. 1B shows the equivalent electrical circuit. FIG. 1C shows the mouse holder enabling mice to receive stimulation while permitting recording of muscle resistance and EMG.

FIGS. 2A-2B depict the effects of 3 sessions of anodal DCS on muscle resistance and EMG response of the stretch reflex in SOD1-G93A mice. FIG. 2A shows an animal in the mouse holder. FIG. 2B (top) shows the muscle resistance and EMG trace recorded before stimulation with anodal msDCS. FIG. 2B (bottom) shows the muscle resistance and EMG trace recorded after 3 sessions of anodal msDCS applied for 50 minutes per day.

FIGS. 3A-3B exhibit the immediate and last effects of anodal msDCS on the EMG response to stretch in SOD1-G93A mice after disease onset. FIG. 3A shows the root-mean-square (green) and raw (pink) EMG traces obtained before and during stimulation with anodal msDCS (1.5 mA at anode, positioned at T9-L6. EMG was recorded from the hind limb triceps surae). Increased muscle activity is visible in the EMG trace after disease onset. The high levels of activity are suppressed immediately at the start of stimulation, with decreased spike amplitude. FIG. 3B shows the root-mean-square (green) and raw (pink) EMG traces obtained after 3 days of stimulation with anodal msDCS with each daily session lasting 50 minutes. The amplitude and frequency of EMG spikes are reduced even when stimulation is not being applied, suggesting lasting effects of the treatment.

FIG. 4 show the immediate effects of anodal msDCS on tremors in the hind limbs of SOD1-G93A mice. ALS mice display tremors and spasms after symptom onset. These tremors can be measured using either a micro-goniometer or a force transducer. FIG. 4 (top) shows tremors in the left hind paw as recorded using a micro-goniometer. FIG. 4 (middle) shows tremors in the right hind paw as recorded with a force transducer. FIG. 4 (bottom) shows the EMG trace of the tremors in the right hind limb triceps surae. In all three plots, the shaded area corresponds to before anodal msDCS is applied. Tremors and spasms are suppressed immediately following start of stimulation on both sides. This enhancement is visible using all methods of measurement.

FIG. 5 displays the lasting effects of anodal msDCS on involuntary muscle contractions and tremors in SOD1-G93A mice after symptom onset. From top to bottom, the figure shows the EMG trace (in blue) and force measurement (in black, recorded with a force transducer), measured before stimulation, during stimulation, after 2 days of stimulation and after 3 days of stimulation. The tremors are immediately suppressed during stimulation. The activity measured after two days of stimulation shows additional reduction of tremors. After 3 days of stimulation, the tremors and muscle spasms are further reduced even without stimulation.

FIG. 6 shows improvements in motor function in SOD1-G93A mice following anodal msDCS. Motor function was evaluated using a modified horizontal ladder scoring system, with a scoring scale of 0-6 (6 is perfect score, with mid-portion of palm of a limb is placed on a rung to bear the animal's full weight) in stimulated and non-stimulated carrier mice as well as non-carrier mice. Mice were video-recorded from below with videos stored and analyzed by investigators blinded to mice treatment. Mice received stimulation for 1 hour per day for 2 days per week, with 1.5 mA delivered through the spinal anode. Stimulation started after disease onset, on average at 106 days of age in this group. Grid walking scores of non-stimulated ALS mice rapidly decline after disease onset (blue), as compared to non-carrier controls (black). In stimulated carrier mice (red), the grid walking scores are significantly higher than scores of unstimulated carrier mice, reflecting enhanced preservation of motor function following stimulation. N=2 non-carriers, 4 stimulated carriers, 2 non-stimulated carriers. Data are presented as mean±SEM.

FIGS. 7A-7F display NKCC1 expression in spinal motor neurons following anodal Ms It will be understood that the msDCS term includes simultaneous direct current stimulation at multiple neuronal targets at different locations on the patient. This may include any combinations of spinal, cortical and peripheral neural stimulation locations. NKCC1 is a neuronal chloride co-transporter involved in the maintenance of chloride gradient. Overexpression of NKCC1 leads to hyperexcitability of the motor neurons. NKCC1 expression was evaluated using immunochemistry in non-carrier mice (FIG. 7A), non-stimulated carrier mice (FIG. 7B), stimulated carrier mice (FIG. 7C), and in the lumbar and cervical motor neurons of a same animal (FIGS. 7D and 7E). NKCC1 expression (in green) is increased in non-stimulated ALS mice as compared to non-carrier control mice and reduced by multi-site anodal DCS. In an animal receiving stimulation, NKCC1 expression is reduced underneath the site of the electrode (lumbar). FIG. 7F shows the relative expression levels of NKCC1 across the three groups.

FIGS. 8A-8F show reduced SOD1 expression in SOD1-G93A mice treated with anodal msDCS. ALS mice overexpress mutant SOD1 (mSOD1) protein, leading to a pathological aggregation. FIGS. 8A-8E are photomicrographs of spinal cords of treated and untreated animals. FIG. 8F shows significantly lower mSDO1 expression in stimulated carrier animals as compared to non-stimulated carriers. N=control: 8 slices from 4 animals; stimulated: 5 slices from 5 animals.

FIG. 9 shows HSP70 expression is reduced in carrier animals as versus non-carrier animals, and is increased in carrier animals after stimulation with anodal msDCS in SOD1-G93A mice. HSP70 is a chaperone protein known to enhance flow of substrates through degradation pathways. HSP70 immunofluorescence intensity of single spinal motor neurons was calculated using ImageJ. HSP70 levels in the spinal cord are significantly reduced in carrier non-stimulated mice (bottom left) as compared to control mice (top left), but it is significantly increased in carrier mice after stimulation (top right). These findings are summarized in a bar graph (bottom right; N=stimulated: 27 motor neurons from 4 animals; control: 19 motor neurons from 3 animals).

FIG. 10 shows the effects of anodal msDCS on levels of phosphorylated tau protein (phTau). Increased phosphorylated tau has been reported in ALS (Stevens et al., “Increased tau phosphorylation in motor neurons from clinically pure sporadic amyotrophic lateral sclerosis patients”, J. Neuropathol. Exp. Neurol., 2019). More phTau is found in non-stimulated carrier mice as compared with non-carriers. Treatment with anodal msDCS reduced phTau levels in carrier mice.

FIG. 11 shows the effects of anodal msDCS on the expression of LC3B in SOD1-G93A mice. LC3B is a protein marker for autophagy activity. The dysregulation of autophagy has been associated with ALS, and decreased LC3B could lead to accelerated motor neuron degeneration following abnormal accumulation of toxic proteins. LC3B expression is increased in carrier mice after stimulation as compared to non-stimulated carrier mice (control).

FIG. 12 exhibits the effects of anodal msDCS on the survival of spinal motor neurons of SOD1-G93A mice. Sibling SD1-G93A mice were separated into stimulated and non-stimulated groups. Stimulated mice received 5 consecutive daily treatments of 60 minutes following disease onset. Mice were sacrificed 10 days after disease onset. Motor neurons were counted after 2 slices were extracted per mouse. Micrographs show 4 sections of the same slice for each example mouse. Stimulation resulted in the preservation of larger alpha motor neurons and an overall 34.8% increase in motor neuron count after stimulation as compared to non-stimulated carriers.

FIG. 13 is a Kaplan-Meier plot showing the effects of anodal msDCS on the survival of SOD1-G93A mice when treatment starts immediately after symptom onset (therapeutic stimulation paradigm). Symptom onset was defined as a score of 80 or less in the grid walking test. Stimulated carrier mice survived for 20.5 days (±2.1 days) on average while non-stimulated carrier mice survived for 11.1 days (±1.2 days) on average, leading to an 84% increase in mean survival time after disease onset for stimulated mice. N=19 stimulated mice; 23 non-stimulated mice. Log Rank (Mantel-Cox), Chi-Square=13.86, p=0.0002; Breslow (Generalized Wilcoxon) Chi-Square=12.25, p=0.0005; Tarone-Ware, Chi-Square=13.12, p=0.0003.

FIG. 14 illustrates an embodiment of a novel approach to treating ALS consisting of a control unit applying msDCS and skin-surface electrodes applied along the spinal column and the peripheral nerves of the four limbs.

FIG. 15 shows an embodiment of a multi-channel unit to be used to sequentially treat an ALS patient from the cervical spinal cord area to the lumbar spinal cord area to slow down ALS disease progression. In one embodiment, each channel is limited to sourcing no more than 5 mA DC with a maximum current density of 0.56 mA/cm² for the smallest electrodes (below safety limits specified by applicable standards).

FIG. 16 illustrates an embodiment of a multi-channel cortical stimulation device utilized to preserve cortical neurons using the present msDCS technology.

FIG. 17 exhibits the effects of anodal msDCS on motor function in transgenic TDP-43 mice. Mice were stimulated for 3 weeks (3 times a week, one hour a day). Stimulated=5 mice; Unstimulated=6 mice. A, Wire hang test. B, Hindlimb grip strength test. C, Grid walking test.

FIG. 18A and FIG. 18B exhibits the effects of anodal msDCS on the formation of TDP-43 protein aggregates in motor neurons of transgenic TDP-43 mice. A, Microscopic images of lumbar SC MNs showing TDP-43 in transgenic and wild-type (WT) mice. In unstimulated mice (n=6), TDP-43 is located inside and outside the nucleus and in stress granules surrounding the nucleus. In stimulated mice (n=5), TDP-43 was only in nucleus, and no stress granules were found. B, Stimulated TDP-43 mice (n=58 MN) have fewer and smaller size particles of TDP-43 outside the nucleus than unstimulated mice (n=71 MN).

FIG. 19 exhibits the kinetics of degradation-pathway associated proteins in the SOD1-G93A model following stimulation with anodal msDCS. Top, Western blots examining the levels of HSP72, HSP70, LC3A and LC3B in control, unstimulated and stimulated SOD1-G93A mice. Mice were 120 days of age. Stimulated and unstimulated mice were showing symptoms. Controls were C57 mice without the SOD1 gene expressed. Mice were stimulated for 1 h, and then sacrificed 24 h or 48 h later-current intensity 0.55 mA per electrode. CTL=control; US=unstimulated; PS24=24 hrs post stim; PS48=48 hrs post stim. Bottom, bar plots showing changes for each protein at 24 hrs and 48 hrs.

### DETAILED DESCRIPTION

The description is not to be taken in a limiting sense, but is made merely for the purpose of illustrating the general principles of these teachings, since the scope of these teachings is best defined by the appended claims.

The above illustrative and further embodiments are described below in conjunction with the following drawings, where specifically numbered components are described and will be appreciated to be thus described in all figures of the disclosure.

As used herein, the singular forms “a,” “an,” and “the” include the plural reference unless the context clearly dictates otherwise.

The term “stimulation,” as used herein, refers to either excitation or inhibition of nerve fibers, also referred to as up regulation or down regulation.

The term “electrical stimulation,” as used here in refers to the production or introduction of current into spinal nerve, neuron, circuit or pathway, whether by applying a voltage or magnetically inducing a current.

Direct Current Stimulation (DCS) is a non-invasive neuromodulation methodology that encompasses using direct current to treat diseases and disorders in mammal, in particular disease and disorders affecting the nervous system of vertebrate beings. DCS includes trans-spinal direct current stimulation (tsDCS), trans-cranial direct current stimulation (tcDCS) and trans-peripheral nerve direct current stimulation (tpnDCS).

Trans-spinal direct current stimulation (tsDCS) is a non-invasive neuromodulation methodology that uses direct current to modulate spinal cord neurons and spinal pathways. tsDCS can induce or suppress expression of specific proteins within the spinal cord neurons. The modulation of expression of specific proteins can impact the manifestations of conditions such as spasticity, hypertonia and dystonia, and can impact the progression of diseases linked to neuronal hyperexcitability.

According to embodiments of the invention, tsDCS includes stimulation of a target spinal location or locations. The stimulation includes applying direct current along a defined current path that includes the target spinal location or locations. The stimulation in one illustrative embodiment is substantially continuous and non-varying at subthreshold level. In another illustrative embodiment the stimulation is varying in whole or in part. In another illustrative embodiment the stimulation includes a combination of varying and non-varying stimulation. In another illustrative embodiment the stimulation includes pulses of stimulation. In yet another illustrative embodiment the stimulation includes pulses of continuous current stimulation where the current flow is not monodirectional.

Trans-cranial direct current stimulation (tcDCS) is a non-invasive neuromodulation methodology that uses direct current to modulate neurons and glial cells of the brain and cortical pathways. tcDCS can induce or suppress expression of specific proteins within neurons and other cells of the brain. The modulation of expression of specific proteins can be therapeutic for treating certain neurological diseases and disorders of the CNS including ALS, other motor neuron disorders, and other neurodegenerative diseases.

According to embodiments of the invention, tcDCS includes stimulation of a target cranial location or locations. The stimulation includes applying direct current along a defined current path that includes the target cranial location or locations. The stimulation in one illustrative embodiment is substantially continuous and non-varying at subthreshold level. In another illustrative embodiment the stimulation is varying in whole or in part. In another illustrative embodiment the stimulation includes a combination of varying and non-varying stimulation. In another illustrative embodiment the stimulation includes pulses of stimulation. In yet another illustrative embodiment the stimulation includes pulses of continuous current stimulation where the current flow is not monodirectional.

Peripheral direct current stimulation (pDCS) is a non-invasive neuromodulation methodology that uses direct current to modulate peripheral nerves. pDCS can induce or suppress expression of specific proteins within the peripheral nerve and surrounding cells, and can affect the ability of the nerve to propagate descending and ascending signals. The modulation of expression of specific proteins can be used in the treatment of certain neurological disorders, including ALS and other motor neuron diseases and neurodegenerative diseases.

According to embodiments of the invention, pDCS includes stimulation of a target peripheral nerve location or locations. The stimulation includes applying direct current along a defined current path that includes the target peripheral nerve location or locations. The stimulation in one illustrative embodiment is substantially continuous and non-varying at subthreshold level. In another illustrative embodiment the stimulation is varying in whole or in part. In another illustrative embodiment the stimulation includes a combination of varying and non-varying stimulation. In another illustrative embodiment the stimulation includes pulses of stimulation. In yet another illustrative embodiment the stimulation includes pulses of continuous current stimulation where the current flow is not monodirectional.

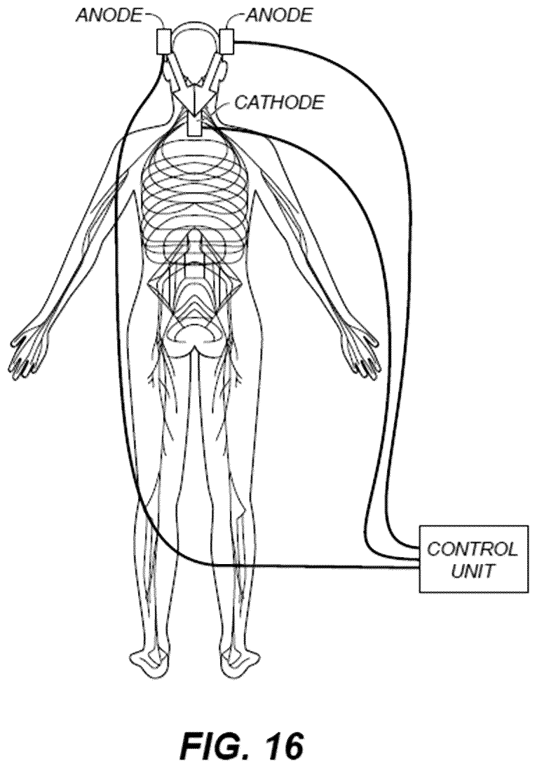

In various practices msDCS refers to the application of direct current stimulation simultaneously at multiple sites along the neural axis, which can include tsDCS, tcDCS and pDCS, and in one case can include a single-circuit stimulation discussed later with respect to FIG. 16.

In practice of the invention, an electrode or array of electrodes is connected to a direct current source and placed at an area of interest, such as either directly over or near the dorsal aspect of the spinal cord, on the cranium or at or near a peripheral nerve. A return electrode or array of electrodes is placed distal therefrom to define a current flow path which in practice can be on the ventral aspect of the body, but not necessarily, directly opposite the electrode located at the area of interest. The direct current is applied to the treatment electrode located at the area of interest as either anode or cathode, depending upon function and desired stimulation.

The following terms may be understood, in the various illustrative by not limiting descriptions of embodiments of invention provided herein, to at least have the following definitions:

“Cathodal stimulation” refers to DCS where the cathode is placed at the desired area of interest for treatment.

“Anodal stimulation” refers to DCS where the anode is placed at the desired area of interest for treatment.

“Spasticity” is defined as the velocity-dependent over-activity of the stretch reflex. Thus, spasticity refers to a condition in which certain muscles are continuously or sporadically contracted. This contraction causes stiffness or tightness of the muscles and can interfere with normal movement, speech and gait, and can be painful. Spasticity is usually caused by damage to a region of the brain or spinal cord. The damage causes a change in the balance of signals between the nervous system and the muscles. This imbalance leads to increased activity in the muscles.

“Hypertonia” refers to impaired ability of damaged motor neurons to regulate descending pathways giving rise to disordered spinal reflexes, increased excitability of muscle spindles, and decreased synaptic inhibition. These consequences result in abnormally increased muscle tone of symptomatic muscles. Hypertonia includes patients exhibiting increased muscle tone in the absence of stretch reflex over-activity, thus distinguishing hypertonia from spasticity.

“Protein expression” refers to the level or amount of a protein or peptide contained within or produced by (e.g. excreted proteins or peptides) cells or tissues. “Differential expression”, differential protein expression” and “differentially expressed” refer to a change in the level or amount of a protein or peptide contained within or produced by cells or tissues. Changes in protein expression can occur in response to specific external signals, such as chemical signals, mechanical signals or direct current stimulation. Such changes in expression can be an increase or a decrease in the level or amount of protein or peptide contained within or produced by cells or tissues. An increase in the level or amount of protein or peptide is also referred to as “up-regulation”, and a decrease in the level or amount of protein or peptide is also referred to as “down-regulation”.

“Messenger RNA expression” (“mRNA expression”) refers to the level or amount of mRNA contained within cells or tissues. “Differential expression”, “differential mRNA expression”, “differential gene expression” and “differentially expressed” refer to a change in the level or amount of mRNA contained within cells or tissues. Changes in mRNA or gene expression can occur in response to specific external signals, such as chemical signals, mechanical signals or direct current stimulation. Such changes in expression can be an increase or a decrease in the level or amount of mRNA contained within cells or tissues. An increase in the level or amount of mRNA is also referred to as “up-regulation”, and a decrease in the level or amount of mRNA is also referred to as “down-regulation”. The terms “mRNA expression” and “gene expression” are used interchangeably throughout this disclosure.

Motor neuron diseases refer to certain neurological diseases that affect motor neurons and include amyotrophic lateral sclerosis, primary lateral sclerosis, progressive muscular atrophy, progressive bulbar palsy, spinal muscular atrophy and post-polio syndrome.

Neurodegenerative diseases refer to certain neurological diseases that result in the progressive loss of neurons and include classic amyotrophic lateral sclerosis, familial SOD1 ALS, progressive muscular atrophy (PMA), frontotemporal lobar degeneration (FTLD), ALS-FTLD, Alzheimer's disease (AD), dementia with Lewy bodies, primary lateral sclerosis (PLS), Parkinson's disease, Huntington's disease, hippocampal sclerosis, limbic-predominant age-related TDP-43 encephalopathy (LATE)/cerebral age-related TDP-43 with sclerosis (CARTS), chronic traumatic encephalopathy (CTE), Perry disease and multisystem proteinopathy.

The present invention supports the hypothesis that the application of msDCS can be beneficial in patients with ALS, as well as other motor neuron diseases and neurodegenerative diseases by both treating symptoms of the disease and slowing disease progression.

Na—K—Cl co-transporter isoform 1 (NKCC1) and K—Cl co-transporter isoform 2 (KCC2) are involved in establishing and maintaining the chloride (Cl⁻) concentration gradient across nerve cell membranes (Misgeld et al., “The role of chloride transport in postsynaptic inhibition of hippocampal neurons”, Science, 1986). Due to the importance of the electrochemical Cl-gradient in determining the strength of inhibition mediated by GABA-A and glycine receptors, an imbalance in protein levels or the activities of NKCC1 and KCC2 has been predicted to lead to hyperexcitability and muscle dysfunction, particularly spasticity (Boulenguez et al., “Down-regulation of the potassium-chloride cotransporter KCC2 contributes to spasticity after spinal cord injury”, Nat. Med., 2010; Mòdol et al., “Prevention of NKCC1 phosphorylation avoids downregulation of KCC2 in central sensory pathways and reduces neuropathic pain after peripheral nerve injury”, Pain, 2014). The mechanism of action underlying the long-term effects of direct current stimulation on spinal or brain excitability is largely unknown. It has been proposed that tsDCS can cause long-term changes in the excitability of spinal cord circuits (Ahmed, “Electrophysiological characterization of spino-sciatic and cortico-sciatic associative plasticity: modulation by trans-spinal direct current and effects on recovery after spinal cord injury in mice”, J. Neurosci., 2013; Ahmed, “Effects of cathodal trans-spinal direct current stimulation on lower urinary tract function in normal and spinal cord injury mice with overactive bladder”, J. Neural. Eng., 2017; Bolzoni and Jankowska, “Presynaptic and postsynaptic effects of local cathodal DC polarization within the spinal cord in anaesthetized animal preparations”, J. Physiol., 2015; Samaddar et al., “Transspinal direct current stimulation modulates migration and proliferation of adult newly born spinal cells in mice”, J. Appl. Physiol., 2016; Song et al., “Combined motor cortex and spinal cord neuromodulation promotes corticospinal system functional and structural plasticity and motor function after injury”, Exp. Neurol., 2016; Wieraszko and Ahmed, “Direct current-induced calcium trafficking in different neuronal preparations”, Neural. Plast., 2016). Our recent findings indicate that direct current stimulation can modulate the expression levels of certain proteins. Therefore, the present invention examined the effects of msDCS that includes tsDCS on changes in protein expression of NKCC1, SOD1, HSP70, LCB3, tau in the spinal cord, as well as examined the effects of msDCS on muscle EMG, tremor, motor function, motor neuron survival and survival of the animals.

A combination of electrophysiology, locomotor analysis and survival studies were used to reveal the influences of msDCS on SOD1-G93A mice. In addition, quantitative real-time PCR (qPCR), Western blotting, and immunohistochemistry were performed to identify changes in protein and/or gene expression of NKCC1, SOD1, tau, TDP-43, HSP70, HSP72, LC3A and LC3B in stimulated spinal tissues from SOD1-G93A or TDP-43 mice.

Illustrative embodiments of the invention include treating motor neuron diseases or neurodegenerative diseases that have been associated with elevated expression of NKCC1, SOD1, TDP-43 or tau. Representative examples of such diseases and disorders include classic amyotrophic lateral sclerosis, familial SOD1 ALS, progressive bulbar palsy, spinal muscular atrophy, post-polio syndrome, progressive muscular atrophy (PMA), frontotemporal lobar degeneration (FTLD), ALS-FTLD, Alzheimer's disease (AD), dementia with Lewy bodies, primary lateral sclerosis (PLS), Parkinson's disease, Huntington's disease, hippocampal sclerosis, limbic-predominant age-related TDP-43 encephalopathy (LATE)/cerebral age-related TDP-43 with sclerosis (CARTS), chronic traumatic encephalopathy (CTE), Perry disease and multisystem proteinopathy.

Illustrative embodiments of the invention include methods of treating ALS and other motor neuron diseases or neurodegenerative diseases comprising the step of delivering non-invasive neuromodulation in a manner that decreases NKCC1 gene expression levels and/or protein expression levels, decreases SOD1 gene expression levels and/or protein expression levels and/or cytosolic protein aggregates, decreases tau gene expression levels and/or protein expression levels and/or cytosolic protein aggregates, decreases TDP-43 gene expression levels and/or protein expression levels and/or cytosolic protein aggregates, or increases HSP70 gene expression levels and/or protein expression levels.

Illustrative embodiments of the invention include methods of treating a disorder in a vertebrate being, including the steps of: applying stimulation between a first electrode and a second electrode of a direct current source with the first electrode being at or proximate to a dorsal aspect of a spinal cord of a vertebrate being or with the first electrode being on a cranium of a vertebrate being; and applying the direct current stimulation at an intensity and for a period of time sufficient to change biological activity of or level of gene expression and/or protein expression and/or cytosolic protein aggregates of NKCC1, SOD1, TDP-43, HSP70, HSP72, tau, LC3A or LC3B; wherein the second electrode is placed at a position remote from the first electrode and the first and second electrodes are oppositely charged, and wherein the direct current is at least one of constant, continuous, pulsed, intermittent, varying and non-varying.

Illustrative embodiments of the invention include methods of treating a disorder in a vertebrate being that are double stimulation methods, including the steps of applying a first stimulation between a first electrode and a second electrode of a direct current source with the first electrode being at or proximate to a dorsal aspect of a spinal cord of a vertebrate being; applying a second stimulation between a third electrode and a fourth electrode of a direct current source with one of the third electrode being at or proximate to a peripheral nerve of a vertebrate being; and applying the direct current stimulation at an intensity and for a period of time sufficient to change biological activity of or level of gene expression and/or protein expression and/or cytosolic protein aggregates of NKCC1, SOD1, TDP-43, HSP70, HSP72, tau, LC3A or LC3B; wherein the second electrode is placed at a position remote from the first electrode and the first and second electrodes are oppositely charged, and the fourth electrode is placed at a position remote from the third electrodes and the third and fourth electrodes are oppositely charged, and wherein the direct current is at least one of constant, continuous, pulsed, intermittent, varying and non-varying.

In some embodiments of the double stimulation method, the first and second stimulations are applied simultaneously or sequentially.

Illustrative embodiments of the invention include methods of treating a disorder in a vertebrate being that are triple stimulation methods, including the steps of applying a first stimulation between a first electrode and a second electrode of a direct current source with the first electrode being at or proximate to a dorsal aspect of a spinal cord of a vertebrate being; applying a second stimulation between a third electrode and a fourth electrode of a direct current source with one of the third electrode being at or proximate to a peripheral nerve of a vertebrate being; and applying the direct current stimulation at an intensity and for a period of time sufficient to change biological activity of or level of gene expression and/or protein expression and/or cytosolic protein aggregates of NKCC1, SOD1, TDP-43, HSP70, HSP72, tau, LC3A or LC3B; wherein the second electrode is placed at a position remote from the first electrode and the first and second electrodes are oppositely charged, and the fourth electrode is placed at a position remote from the third electrodes and the third and fourth electrodes are oppositely charged, and wherein the direct current is at least one of constant, continuous, pulsed, intermittent, varying and non-varying; and further comprising applying a third stimulation between a fifth electrode and a sixth electrode of a direct current source with the fifth electrode being on a cranium of a vertebrate being; and wherein the sixth electrode is placed at a position remote from the fifth electrode and the fifth and sixth electrodes are oppositely charged.

In some embodiments, of the triple stimulation method the first, second and third stimulations are applied simultaneously or sequentially.

In illustrative examples of the invention where peripheral nerves are stimulated, the peripheral nerve innervates a skeletal muscle. Representative examples of some peripheral nerves include leg nerves or arm nerves including, but not limited to a sciatic nerve, a peroneal nerve, a plantar digital nerve, a femoral nerve, a saphenous nerve, a sural nerve, a tibial nerve, a median nerve, a musculocutaneous nerve, a palmar digital nerve, a radial nerve, and an ulnar nerve.

In some embodiments of the single, double and triple stimulation methods, the stimulations are performed over a period of time that includes a series of stimulation sessions on 1 or more days, the days being consecutive or non-consecutive.

In some embodiments of the single, double and triple stimulation methods, the disorder being treated is a muscle tone disorder such as spasticity, spasticity following spinal cord injury, hypertonia and dystonia.

In some embodiments of the single, double and triple stimulation methods, the biological activity of or the level of gene expression and/or protein expression of NKCC1 is decreased, the biological activity of or the level of gene expression and/or protein expression, and/or cytosolic protein aggregates of SOD1 is decreased, the biological activity of or the level of gene expression and/or protein expression, and/or cytosolic protein aggregates of TDP-43 is decreased, the biological activity of or the level of gene expression and/or protein expression and/or cytosolic protein aggregates of tau is decreased, the biological activity of or the level of gene expression and/or protein expression of HSP70 or HSP72 is increased, or the biological activity of or the level of gene expression and/or protein expression of LC3A or LC3B is increased.

Illustrative embodiments of the invention include methods of treating ALS in a vertebrate being. The non-invasive electrical stimulation methods described herein are designed to halt the progression of ALS by protecting motor and cortical neurons from dying, i.e. the methods described herein prolong neuronal cell life or at least slow down the rate of motor and cortical neuron cell death. Without intending to be bound by theory, it is believed that either suppressing NKCC1, SOD1, TDP-43 or tau expression and/or activity or cytosolic protein aggregates of, or increasing HSP70, HSP72, LC3A or LC3B expression and/or activity in spinal and cortical neurons will lead to a slowing or halt in the progression of ALS by prolonging motor and cortical neuronal cell life.

In some embodiments, the methods of treating ALS include the steps of: applying stimulation between an A electrode and a B electrode of a direct current source with the A electrode being at or proximate to a dorsal aspect of a spinal cord of a vertebrate being or with the A electrode being on a cranium of a vertebrate being; and applying the direct current stimulation at an intensity and for a period of time sufficient to prolong neuronal cell life associated with ALS disease state; wherein the B electrode is placed at a position remote from the A electrode and the A and B electrodes are oppositely charged, and wherein the direct current is at least one of constant, continuous, pulsed, intermittent, varying and non-varying.

It is envisioned that, when treating ALS patients, multiple spinal cord locations along the length of the spinal cord will be treated in order to prolong neuronal cell life at multiple locations. Accordingly, in some embodiments, the methods of treating ALS include a plurality of A electrodes located at or proximate to a plurality of positions along the dorsal aspect of the spinal cord and a plurality of B electrodes placed at positions remote from the plurality of A electrodes. In other embodiments, one set of A and B electrodes is used, and the electrodes are moved to different locations along the length of the spinal cord in a series of treatments.

It is also envisioned that, when treating ALS patients, multiple regions of the brain associated with movement control will be treated including the motor cortex (Area 6 and Area 4, also known as the primary motor cortex), basal ganglia and the cerebellum. Accordingly, in some embodiments, the methods of treating ALS include a plurality of A electrodes located at a plurality of positions on the cranium associated with movement control and a plurality of B electrodes are placed at positions remote from the plurality of A electrodes. In other embodiments, one set of A and B electrodes is used, and the electrodes are moved to different cranial positions associated with movement control in a series of treatments.

In some embodiments of the methods of treating ALS, the stimulation applied between the plurality of A and B electrodes is applied simultaneously or sequentially.

In some embodiments, the methods of treating ALS patients include the steps of: applying a first stimulation between an A electrode and a B electrode of a direct current source with the A electrode being at or proximate to a dorsal aspect of a spinal cord of a vertebrate being; applying a second stimulation between a C electrode and a D electrode of a direct current source with the C electrode being on a cranium of a vertebrate being; and applying the direct current stimulation at an intensity and for a period of time sufficient to prolong neuronal cell life; wherein the B electrode is placed at a position remote from the A electrode and the A and B electrodes are oppositely charged, and the D electrode is placed at a position remote from the C electrode and the C and D electrodes are oppositely charged, and wherein the direct current is constant or pulsed. In some embodiments, the first and second stimulations are applied simultaneously or sequentially.

In some embodiments, the methods of treating ALS include a plurality of A electrodes located at or proximate to a plurality of positions along the dorsal aspect of the spinal cord and a plurality of B electrodes placed at positions remote from the plurality of A electrodes. In other embodiments, one set of A and B electrodes is used, and the electrodes are moved to different locations along the length of the spinal cord in a series of treatments.

In some embodiments, the methods of treating ALS include a plurality of C electrodes located at a plurality of positions on the cranium associated with movement control and a plurality of D electrodes are placed at positions remote from the plurality of C electrodes. In other embodiments, one set of C and D electrodes is used, and the electrodes are moved to different cranial positions associated with movement control in a series of treatments.

In some embodiments, the stimulation applied between the plurality of A and B electrodes and between the plurality of C and D electrodes is applied simultaneously or sequentially.

In some embodiments, the methods of treating ALS further include applying a third stimulation between an E electrode and an F electrode of a direct current source with the E electrode being at or proximate to a peripheral nerve of a vertebrate being; and wherein the F electrode is placed at a position remote from the E electrode and the E and F electrodes are oppositely charged. In some embodiments the first, second and third stimulations are applied simultaneously or sequentially.

In some embodiments, the methods of treating ALS include a plurality of E electrodes located at or proximate to a plurality of positions along a peripheral nerve or are located at or proximate to a plurality of peripheral nerves and a plurality of F electrodes are placed at positions remote from the plurality of E electrodes. In other embodiments, one set of C and D electrodes is used, and the electrodes are moved to different positions along a peripheral nerve or are moved to different peripheral nerves. In some embodiments, the stimulation applied between the plurality of E and F electrodes is applied simultaneously or sequentially. In some embodiments where peripheral nerves are stimulated, the peripheral nerve innervates a skeletal muscle. Representative examples of some peripheral nerves are disclosed above.

In some embodiments of the methods of treating ALS, the period of time comprises a series of stimulation sessions on 1 or more days, the days being consecutive or non-consecutive.

In some embodiments of the methods of treating ALS, the method includes applying the direct current stimulation at an intensity and for a period of time sufficient to change biological activity of or level of gene expression and/or protein expression of NKCC1, SOD1, TDP-43, tau, HSP70, HSP72, LC3A or LC3B. In some embodiments, the biological activity of or the level of gene expression and/or protein expression of NKCC1, SOD1, TDP-43 or tau is decreased, while in other embodiments, the biological activity of or the level of gene expression and/or protein expression of HSP70, HSP72, LC3A or LC3B is increased.

In some embodiments, we use multiple spinal electrodes (e.g., anode) each with its own constant current source. This divides the current and delivers more even stimulation as compared to one large rectangular spinal electrode.

In other embodiments, multiple spinal electrodes (e.g., anode) each with its own constant current source, are combined with a capability for bilateral peripheral stimulation by using additional constant current sources.

In another embodiment we also include simultaneous cortical stimulation.

In practices of embodiments of the invention, cervical and lumbar stimulation treatments are conducted on different days, e.g., upper limbs on one day, lower limbs the following day.

Animals, particularly mammals including humans, are the subjects of the DCS treatments discussed herein. In illustrative and non-limiting embodiments, treatment of humans in practice of the invention can include application of DCS, for example, generally within a range of over 1 mA and under 6 mA, and more particularly within a range of about 3.5-4 mA, and can be applied for about 20-60 min/day. In other illustrative and non-limiting embodiments, treatments of humans in practice of the invention can include application of trans-cranial DCS (tcDCS) or peripheral DCS (pDCS). DCS treatment can be as often as indicated on a scheduled day or alternating days, or any other treatment regime intended to affect repair or recovery.

In practicing the methods of the invention disclosed herein, the following systems or devices are used.

Illustrative embodiments of the invention include a system for treatment of ALS or other motor neuron diseases in a vertebrate being, the system including: a first stimulation component configured to provide peripheral direct current stimulation of a peripheral nerve associated with motor neuron disease in a vertebrate being; the first stimulation component including a neural stimulation circuit having neural stimulation poles configured to stimulate said peripheral nerve; a second stimulation component configured to provide spinal direct current stimulation at a spinal location associated with regulation of said peripheral nerve, said second stimulation component defining a spinal stimulation circuit having an active spinal stimulation pole and a spinal reference pole, said spinal stimulation circuit configured to provide constant-current trans-spinal direct current stimulation between said spinal stimulation pole and said spinal reference pole for stimulating said spinal location; the active spinal stimulation pole being relatively proximal to said spinal location; the spinal reference pole being relatively distal to said spinal location; and a controller component configured to ensure that said active spinal pole and said proximal neural pole are excited at opposite polarities, forming a resulting polarization circuit, said resulting polarization circuit being configured to provide a polarizing current flow between said active spinal pole and said proximal neural pole according to said opposite polarities, for changing biological activity of or level of gene expression and/or protein expression of a target molecule according to said polarizing current flow; said controller component being also configured to provide peripheral direct current stimulation and spinal direct current stimulation for a predetermined time period and repeat stimulation a predetermined number of times over a predetermined number of days.

In some embodiments of the system, the controller component is configured to provide said current flow including at least one of constant, continuous, pulsed, intermittent, varying and non-varying current flow.

In some embodiments of the system, the controller component is further configured to simultaneously control the range of current supplied by the first and second stimulation components.

In some embodiments of the system, the first stimulation component includes positive and negative poles for providing stimulation current to stimulation electrodes disposed for stimulation of said peripheral nerve, said positive and negative poles disposed for one electrode operatively connected to the positive pole and another electrode operatively connected to the negative pole. In some embodiments of the system, the second stimulation component includes positive and negative poles for providing stimulation current to stimulation electrodes disposed for delivering stimulation across said spinal location, said positive and negative poles disposed for one electrode operatively connected to the positive pole and another electrode operatively connected to the negative pole.

In some embodiments of the system, at least one of the stimulation electrodes is implanted.

In yet other embodiments of the system, at least one of the controller component and an electrical source are disposed in a wearable housing.

In some embodiments of the system, the predetermined time period, the predetermined number of times and the predetermined number of days are selected to change biological activity of or level of gene expression and/or protein expression of NKCC1, SOD1, TDP-43, tau, HSP70, HSP72, LC3A or LC3B. In certain embodiments of the system, the biological activity of or the level of gene expression and/or protein expression of NKCC1, SOD1, TDP-43 or tau is decreased, while other embodiments, the biological activity of or the level of gene expression and/or protein expression of HSP70, HSP72, LC3A or LC3B is increased.

Illustrative embodiments of the invention include a stimulation device for regulating biological activity associated with one of the spinal cord or the brain, comprising: a direct current voltage source having a plurality of terminals; a first of the terminals for connecting a first electrode to the direct current voltage source; the first electrode being at one of a dorsal aspect of a spinal cord of a vertebrate being or on a cranium of a vertebrate being; a second of the terminals for connecting a second electrode to the direct current voltage source; the second electrode being placed at a position remote from the first electrode; the first and second electrodes being oppositely charged; and a controller component configured to control of current flow between the electrodes; the controller component being also configured to provide direct current stimulation for a predetermined time period and repeat stimulation a predetermined number of times over a predetermined number of days.

In some embodiments of the stimulation device, the controller component is configured to provide said current flow including at least one of constant, continuous, pulsed, intermittent, varying, and non-varying current flow.

In some embodiments of the stimulation device, at least one of the first and second electrodes is implanted.

In yet other embodiments of the stimulation device, at least one of the controller component and the direct current voltage source are disposed in a wearable housing.

In some embodiments of the stimulation device, the predetermined time period, the predetermined number of times and the predetermined number of days are selected to change biological activity of or level of gene expression and/or protein expression or cytosolic protein aggregation of NKCC1, SOD1, TDP-43, tau, HSP70, HSP72, LC3A or LC3B.

Illustrative embodiments of the invention include systems for treatment of ALS in a vertebrate being, the system including: a plurality of A stimulation components configured to provide peripheral direct current stimulation of a peripheral nerve at a plurality of locations along the peripheral nerve or to provide peripheral direct current stimulation of a plurality of peripheral nerves in a vertebrate being; each of said A stimulation components including a neural stimulation circuit having neural stimulation poles configured to stimulate said peripheral nerve or plurality of peripheral nerves; a plurality of B stimulation components configured to provide spinal direct current stimulation at a plurality of spinal locations associated with regulation of said peripheral nerve or plurality of peripheral nerves, each of said B stimulation components defining a spinal stimulation circuit having an active spinal stimulation pole and a spinal reference pole, said spinal stimulation circuit configured to provide constant-current trans-spinal direct current stimulation between said spinal stimulation pole and said spinal reference pole for stimulating said spinal location; the active spinal stimulation pole being relatively proximal to said spinal location; the spinal reference pole being relatively distal to said spinal location; and a controller component configured to ensure that said active spinal pole and said proximal neural pole are excited at opposite polarities, forming a resulting polarization circuit, said resulting polarization circuit being configured to provide a polarizing current flow between said active spinal pole and said proximal neural pole according to said opposite polarities, for changing biological activity of or level of gene expression and/or protein expression of a target molecule according to said polarizing current flow; said controller component being also configured to provide peripheral direct current stimulation and spinal direct current stimulation for a predetermined time period and repeat stimulation a predetermined number of times over a predetermined number of days.

In some embodiments of the system, the controller component is configured to provide said current flow including at least one of constant, continuous, pulsed, intermittent, varying and non-varying current flow.

In some embodiments of the system, the controller component is further configured to simultaneously control the range of current supplied by the A and B stimulation components.

In some embodiments of the system, the A stimulation components include positive and negative poles for providing stimulation current to stimulation electrodes disposed for stimulation of said plurality of locations of a peripheral nerve or said plurality of peripheral nerves, said positive and negative poles disposed for one electrode operatively connected to the positive pole and another electrode operatively connected to the negative pole.

In some embodiments of the system, the B stimulation components include positive and negative poles for providing stimulation current to stimulation electrodes disposed for delivering stimulation across said plurality of spinal locations, said positive and negative poles disposed for one electrode operatively connected to the positive pole and another electrode operatively connected to the negative pole.

In some embodiments of the system, at least one of the stimulation electrodes is implanted.

In yet other embodiments of the system, at least one of the controller component and an electrical source are disposed in a wearable housing.

In some embodiments of the system, the predetermined time period, the predetermined number of times and the predetermined number of days are selected to change biological activity of or level of gene expression and/or protein expression of NKCC1, SOD1, TDP-43, tau, HSP70, HSP72, LC3A or LC3B. In certain embodiments of the system, the biological activity of or the level of gene expression and/or protein expression or cytosolic protein aggregation of NKCC1, SOD1, TDP-43 or tau is decreased. In certain embodiments of the system, the biological activity of or the level of gene expression and/or protein expression of HSP70, HSP72, LC3A or LC3B is increased.

Illustrative embodiments of the invention include a stimulation device for treatment of ALS, comprising: a direct current voltage source having a plurality of terminals; a plurality of A terminals for connecting a plurality of A electrodes to the direct current voltage source; the plurality of A electrodes being at a plurality of locations of a dorsal aspect of a spinal cord of a vertebrate being or at a plurality of locations on a cranium of a vertebrate being; a plurality of B terminals for connecting a plurality of B electrodes to the direct current voltage source; the plurality of B electrodes being placed positions remote from the plurality of A electrodes; the A and B electrodes being oppositely charged; and a controller component configured to control of current flow between the electrodes; the controller component being also configured to provide direct current stimulation for a predetermined time period and repeat stimulation a predetermined number of times over a predetermined number of days.

In some embodiments of the stimulation device, the controller component is configured to provide said current flow including at least one of constant, continuous, pulsed, intermittent, varying, and non-varying current flow.

In some embodiments of the stimulation device, at least one of the A and B electrodes is implanted.

In yet other embodiments of the stimulation device, at least one of the controller component and the direct current voltage source are disposed in a wearable housing.

In some embodiments of the stimulation device, the predetermined time period, the predetermined number of times and the predetermined number of days are selected to change biological activity of or level of gene expression and/or protein expression or cytosolic protein aggregation of NKCC1, SOD1, TDP-43, tau, HSP70, HSP72, LC3A or LC3B. In some embodiments of the stimulation device, the biological activity of or the level of gene expression and/or protein expression or cytosolic protein aggregation of NKCC1, SOD1, TDP-43 or tau is decreased. In some embodiments of the stimulation device, the biological activity of or the level of gene expression and/or protein expression of HSP70, HSP72, LC3A or LC3B is increased.

The systems and stimulation devices of the present invention are further described below with reference to the figures.

Although the above described embodiments illustrate a pair of electrodes being applied by each stimulation components, embodiments in which a plurality of pair of electrodes are applied with one stimulation component are also within the scope of these teachings. For example, in some embodiments, a plurality of pair of electrodes are applied with the first stimulation component, where one of each pair of electrodes is applied to a different spinal or cranial location.

While these teachings have been described in terms of specific embodiments, it is evident in view of the foregoing description that numerous alternatives, modifications and variations will be apparent to those skilled in the art. Accordingly, these teachings are intended to encompass all such alternatives, modifications and variations which fall within the scope and spirit of the present teachings and the following claims.

### EXAMPLES

### Example 1: Materials and Methods

### Animals

Transgenic SOD1-G93A mice were used for all of the animal studies performed from a breeding colony established at CUNY/CSI. The transgenic SOD1-G93A mouse is the most widely-used murine model of ALS and is engineered to overexpress a mutant form of human Cu/Zn superoxide dismutase 1 (SOD1). The mice develop muscle tremors, paralysis, premature death and exhibit motor neuron loss. Mice are assigned to specific group based on genotyping and copy number at 60 days of age (pre-symptomatic). qPCR is used to quantify the number of copies in carrier animals. The inclusion of female and male groups is based on publications which found that female and male mice (G93A) have different survival rates. For therapeutic stimulation experiments, carrier animals were divided into 2 groups: 1) Anodal msDCS-treated group; and 2) Carrier unstimulated group. Treatment starts on the first day of confirmed disease and signs of motor dysfunction (early stage). This is confirmed using a grid walking test. Animals are allowed to walk 30 steps and videotaped from the underside. Videos were analyzed by a blinded investigator. We used a 6-point foot fault score system to quantify the grid walking. Since grid walking is very sensitive to spinal or cortical dysfunction (Ruegsegger, “Aberrant association of misfolded SOD1 with Na(+)/K(+) ATPase-α3 impairs its activity and contributes to motor neuron vulnerability in ALS”, Acta. Neuropathol., 2016), this system was found to be very accurate in detecting early stages of disease in mice (preliminary data). All of the study protocols were approved by the College of Staten Island IACUC committee.

### Illustrative Circuit and Power Source

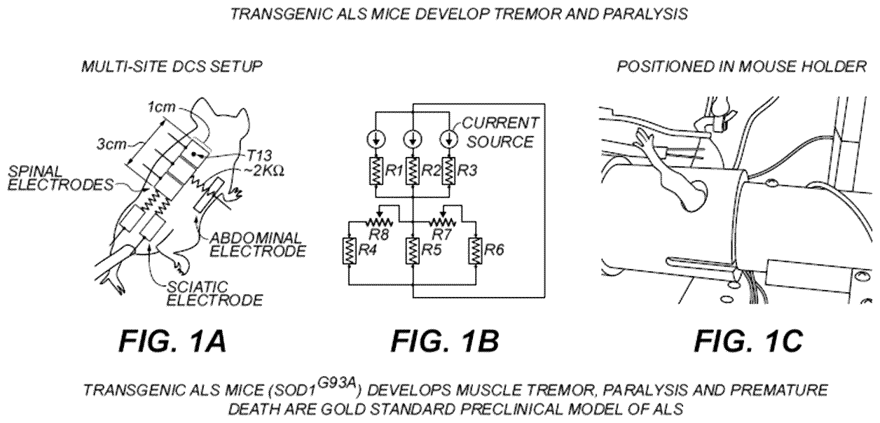

Passing current to peripheral nerves is required to attenuate and modulate motor neuron hyperexcitability. Moreover, the direction and distribution of this current can be regulated. The msDCS protocols of the present invention employed a circuit to non-invasively pass direct current (DC) to the spinal cord and the sciatic nerves of the affected limbs by using over-skin electrodes as shown in FIG. 1A. The reference current source delivered the spinal current divided through three spinal electrodes. To prevent evoking nerve activity, the current passing from the spinal electrodes through to the sciatic nerve electrodes was attenuated by dividing the spinal current into three branches: the first branch connected directly to the abdominal electrode and carried un-attenuated current, while the second and third branches passed through 300 kΩ resistors to attenuate current to the sciatic nerves. A schematic of the circuit is shown in FIG. 1B. Monitoring of current parameters and verification of DC attenuation was performed at the beginning, during, and upon completion of each usage employing a bench-top digital multi-meter.

### Mouse Holder

A mouse restraining system was fabricated in our laboratory (FIG. 1C) from three components. 1) A clear Plexiglas acrylic tube served as the mouse holding chamber. 2) An internally adjustable support system made of a clear acrylic concave stabilization plate. The location of the plate was able to be adjusted linearly along the length of the chamber via a handle that protruded through a cutout that ran the entire length of the tube. In addition, the concave surface of the plate was designed to contour to the back of the animal, thereby securing it dorsally. This surface could also be adjusted externally to accommodate various sizes of mice. 3) Four over-skin stimulating electrodes with each composed of a wick-covered 1 cm×1.5 cm stainless-steel plate. One of the electrodes was permanently fixed to the floor of the holding chamber (for the abdominal reference electrode), while a second electrode was permanently fixed to the middle of the stabilization plate (for the dorsal active spinal electrode) and the location of this plate could be adjusted. The remaining two electrodes could be adjusted linearly by sliding them along cutouts present on the long sides of the stabilization plate, and these served as left and right sciatic nerve conductors, respectively. The abdominal surface of the holding chamber had two openings, one for each of the animal's hind limbs. Each opening was equipped with a knee stabilizer pad to ensure full knee extension during testing. Prior to testing, a cap with an opening in the center was placed on the anterior end of the holding chamber for breathing and for isoflurane administration. To limit hind limb movement to the ankle joint during stretching, another acrylic stand was created to secure the distal leg. The stand had an adjustable stainless-steel ankle stabilizer clamp that could be moved in the x, y, and z axes to achieve proper alignment of the foot under the presser, and to adjust the hip angle of the animal before stretching.

### Example 2: Anodal msDCS Results in Suppression of EMG Activity

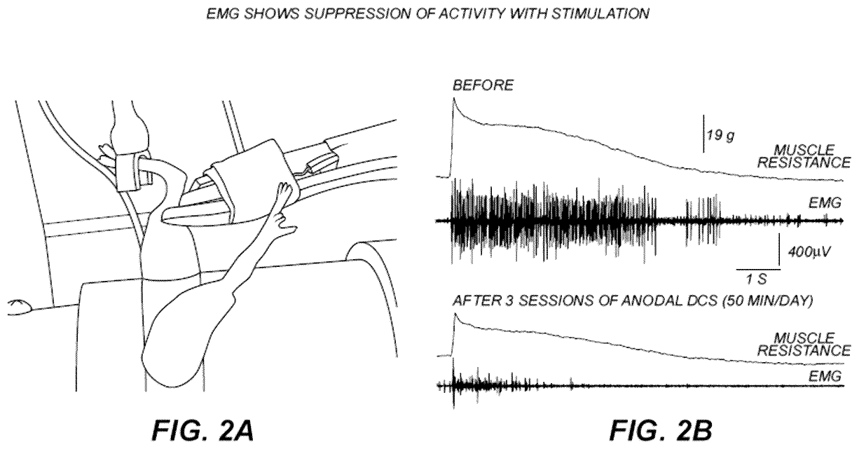

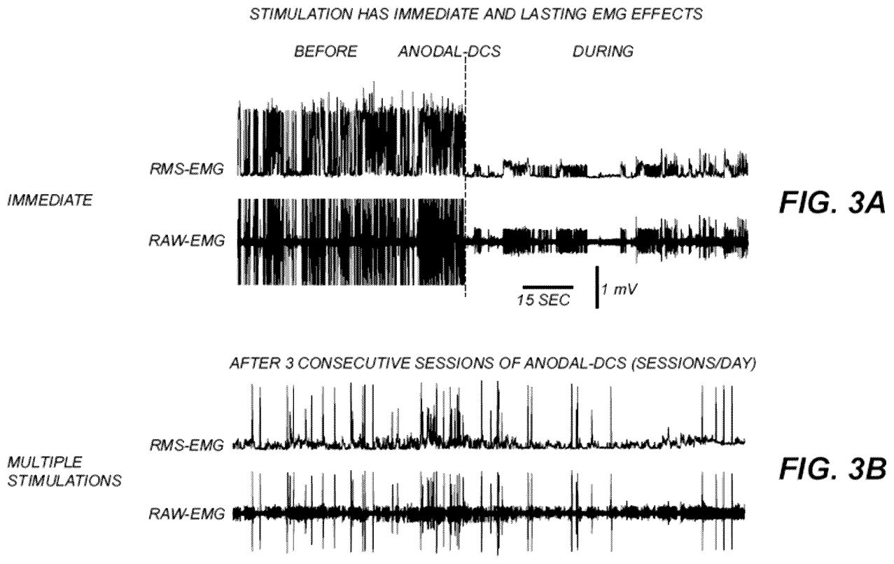

The immediate and lasting effects of anodal msDCS on the EMG response to stretch in SOD1-G93A mice after disease onset is shown in FIGS. 2A-2B and 3A-3B. FIGS. 2A-2B depict the effects of 3 sessions of anodal DCS on muscle resistance and EMG response of the stretch reflex in SOD1-G93A mice. FIG. 2A shows an animal in the mouse holder. FIG. 2B (top) shows the muscle resistance and EMG trace recorded before stimulation with anodal msDCS (1.5 mA). FIG. 2B (bottom) shows the muscle resistance and EMG trace recorded after 3 sessions of anodal msDCS applied for 50 minutes per day. FIG. 3A shows the root-mean-square (green) and raw (pink) EMG traces obtained before and during stimulation with anodal msDCS (1.5 mA at anode, positioned at T9-L6. EMG was recorded from the hind limb triceps surae). Increased muscle activity is visible in the EMG trace after disease onset. The high levels of activity are suppressed immediately at the start of stimulation, with decreased spike amplitude. FIG. 3B shows the root-mean-square (green) and raw (pink) EMG traces obtained after 3 days of stimulation with anodal msDCS with each daily session lasting 50 minutes. The amplitude and frequency of EMG spikes are reduced even when stimulation is not being applied, suggesting lasting effects of the treatment.

### Example 3: Anodal msDCS Results in Suppression of Tremors

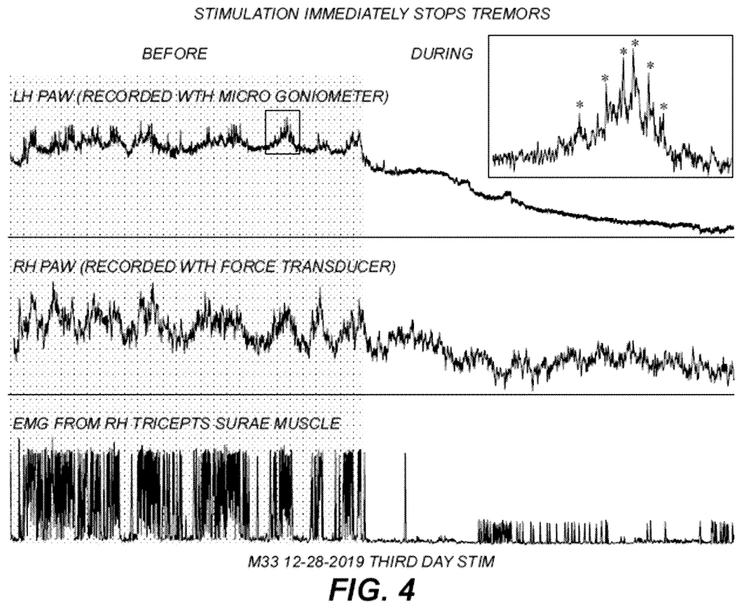

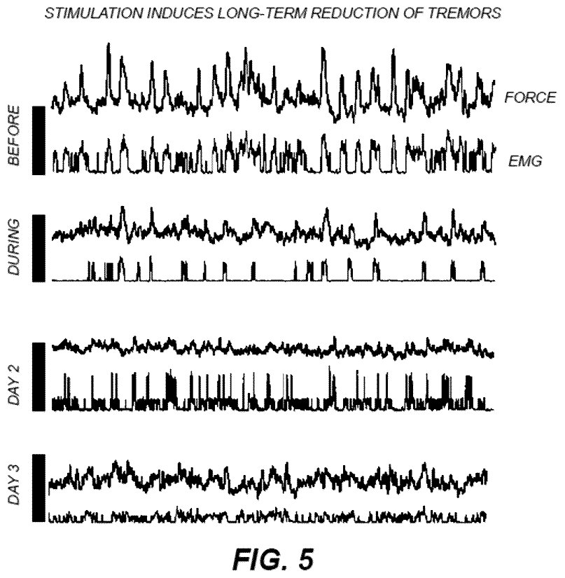

ALS mice display tremors and spasms after symptom onset. These tremors can be measured using either a micro-goniometer or a force transducer. FIG. 4 show the immediate effects of anodal msDCS on tremors in the hind limbs of SOD1-G93A mice. FIG. 4 (top) shows tremors in the left hind paw as recorded using a micro-goniometer. FIG. 4 (middle) shows tremors in the right hind paw as recorded with a force transducer. FIG. 4 (bottom) shows the EMG trace of the tremors in the right hind limb triceps surae. In all three plots, the shaded area corresponds to before anodal msDCS is applied. Tremors and spasms are suppressed immediately following start of stimulation on both sides. This enhancement is visible using all methods of measurement. FIG. 5 displays the lasting effects of anodal msDCS on involuntary muscle contractions and tremors in SOD1-G93A mice after symptom onset. From top to bottom, the figure shows the EMG trace (in blue) and force measurement (in black, recorded with a force transducer), measured before stimulation, during stimulation, after 2 days of stimulation and after 3 days of stimulation. The tremors are immediately suppressed during stimulation. The activity measured after two days of stimulation shows additional reduction of tremors. After 3 days of stimulation, the tremors and muscle spasms are further reduced even without stimulation.

### Example 4: Anodal msDCS Results in Improvement of Motor Function

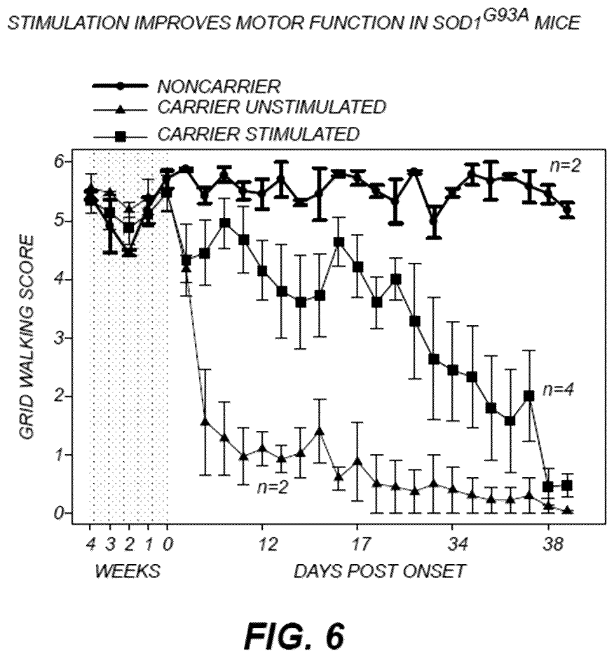

Motor function was evaluated using a modified horizontal ladder scoring system, with a scoring scale of 0-6 (6 is perfect score, with mid-portion of palm of mouse limb placed on a rung to bear the animal's full weight) in stimulated and non-stimulated carrier mice as well as non-carrier mice. Mice were video-recorded from below with videos stored and analyzed by investigators blinded to mice treatment. FIG. 6 shows improvements in motor function in SOD1-G93A mice following anodal msDCS. Mice received stimulation for 1 hour per day for 2 days per week, with 1.5 mA delivered through the spinal anode. Stimulation started after disease onset, on average at 106 days of age in this group. Grid walking scores of non-stimulated ALS mice rapidly decline after disease onset (blue), as compared to non-carrier controls (black). In stimulated carrier mice (red), the grid walking scores are significantly higher than scores of unstimulated carrier mice, reflecting enhanced preservation of motor function following stimulation. N=2 non-carriers, 4 stimulated carriers, 2 non-stimulated carriers. Data are presented as mean±SEM.

### Example 5: Anodal msDCS Reduces Expression of NKCC1

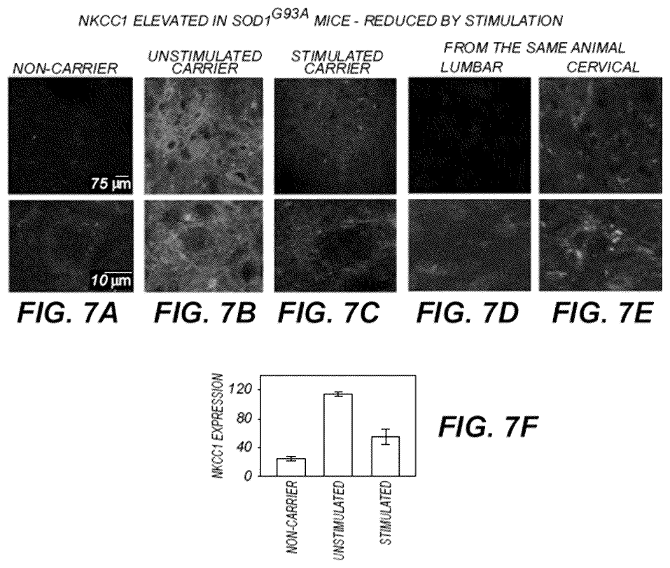

NKCC1 is a neuronal chloride co-transporter involved in the maintenance of chloride gradient. Overexpression of NKCC1 leads to hyperexcitability of the motor neurons. FIGS. 7A-7F display NKCC1 expression in spinal motor neurons following anodal msDCS in SOD1-G93A mice. NKCC1 expression was evaluated using immunochemistry in non-carrier mice (FIG. 7A), non-stimulated carrier mice (FIG. 7B), stimulated carrier mice (FIG. 7C), and in the lumbar and cervical motor neurons of a same animal (FIGS. 7D and 7E). NKCC1 expression (in green) is increased in non-stimulated ALS mice as compared to non-carrier control mice and reduced by multi-site anodal DCS. In an animal receiving stimulation, NKCC1 expression is reduced underneath the site of the electrode (lumbar). FIG. 7F shows the relative expression levels of NKCC1 across the three groups.

### Example 6: Anodal msDCS Reduces Expression of SOD1 Protein

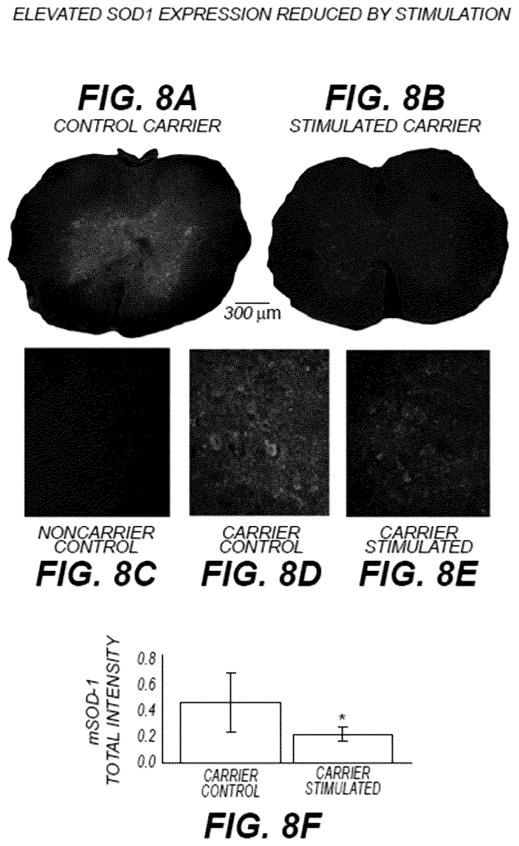

Aggregation of SOD1 is a pathological feature of a subset of familial ALS (Paré et al., “Misfolded SOD1 pathology in sporadic amyotrophic lateral sclerosis”, and the transgenic SOD1-G93A mouse model overexpresses mutant SOD1 (mSOD1) protein, leading to a pathological aggregation. FIGS. 8A-8F show reduced SOD1 expression in SOD1-G93A mice treated with anodal Multi-Site DCS. FIGS. 8A-8E are photomicrographs of spinal cords of treated and untreated animals. FIG. 8F shows significantly lower mSDO1 expression in stimulated carrier animals as compared to non-stimulated carriers. N=control: 8 slices from 4 animals; stimulated: 5 slices from 5 animals.

### Example 7: Anodal msDCS Induces an HSP70 Response

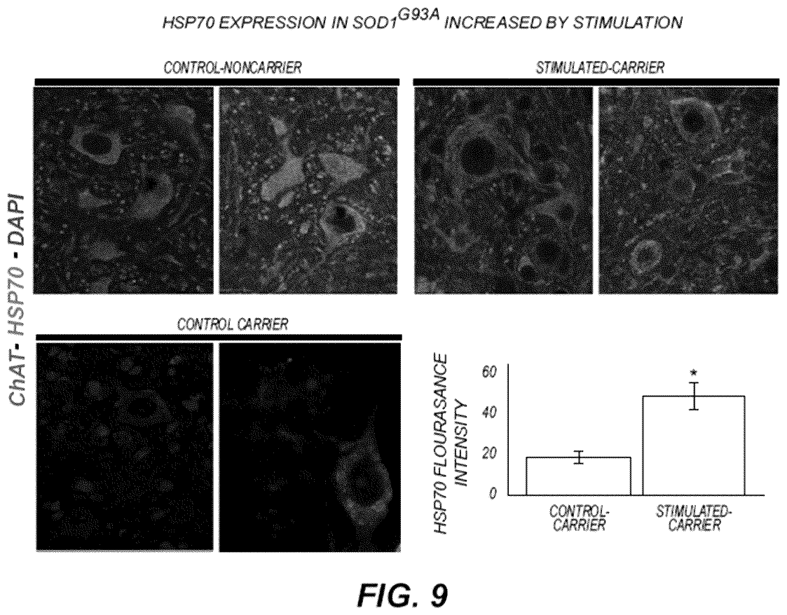

HSP70 and HSP72 are chaperone proteins known to enhance flow of substrates through degradation pathways. FIG. 9 shows HSP70 expression is reduced in carrier animals as versus non-carrier animals, and is increased in carrier animals after stimulation with anodal msDCS in SOD1-G93A mice. HSP70 immunofluorescence intensity of single spinal motor neurons was calculated using ImagJ. HSP70 levels in the spinal cord are significantly reduced in carrier non-stimulated mice (bottom left) as compared to control mice (top left), but it is significantly increased in carrier mice after stimulation (top right). These findings are summarized in a bar graph (bottom right; N=stimulated: 27 motor neurons from 4 animals; control: 19 motor neurons from 3 animals).

### Example 8: Anodal msDCS Reduces Expression of Phosphorylated Tau

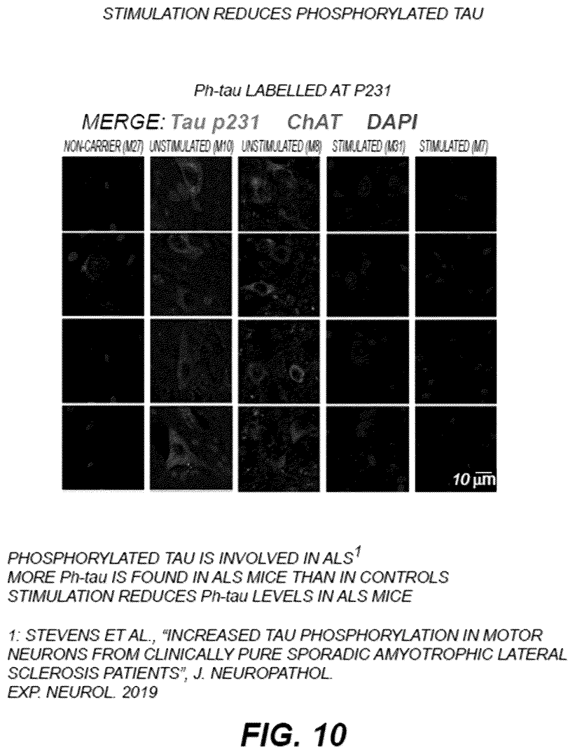

Increased phosphorylated tau protein (phTau) has been reported in ALS. FIG. 10 shows the effects of anodal msDCS on levels of phosphorylated tau protein (phTau). There is a higher level of phTau found in non-stimulated carrier mice as compared with non-carriers. Treatment with anodal msDCS reduced phTau levels in carrier mice.

### Example 9: Anodal msDCS Induces Increased Expression of LC3B

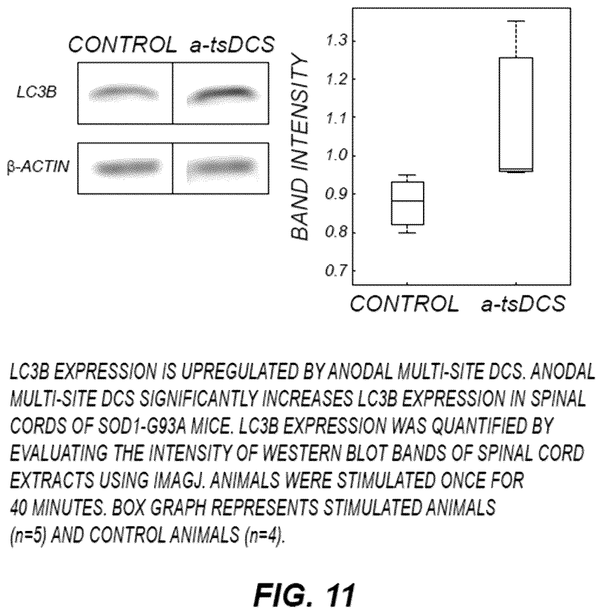

We investigated the expression of LC3B (microtubule-associated protein-1 light chain 3 beta) in SOD1-G93A mice. LC3B and LC3A are protein markers for autophagy activity. Autophagy dysregulation has been associated with ALS and decreased LC3B or LC3A could lead to accelerated motor neuron degradation following abnormal accumulation of toxic proteins. FIG. 11 shows the effects of anodal msDCS on the expression of LC3B in SOD1-G93A mice. LC3B expression is increased in carrier mice after stimulation as compared to non-stimulated carrier mice (control).

### Example 10: Kinetics of Degradation Pathway-Associated Proteins in SOD1-G93A Mice

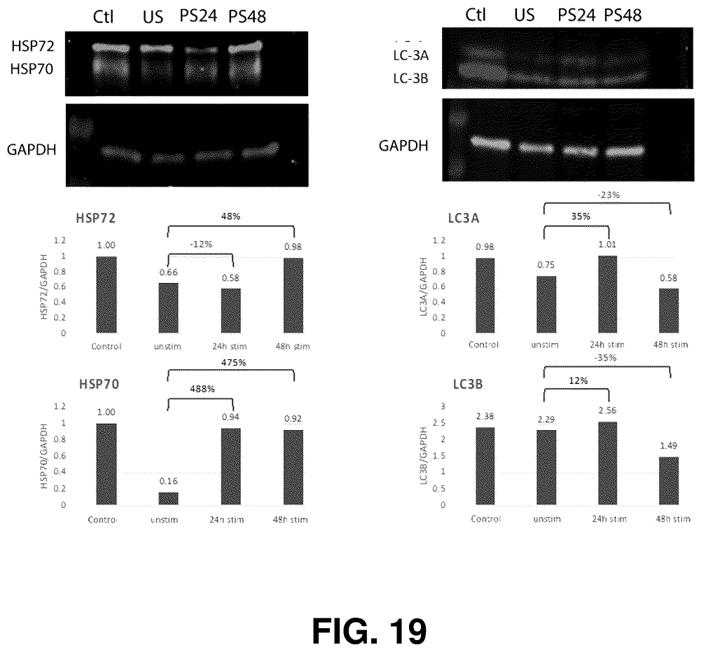

We examined the kinetics of changes in proteins associated with degradation pathways in SOD1-G93A mice. These proteins included HSP72, HSP70, LC3A and LC3B. FIG. 19 shows the effects of anodal msDCS on the expression of HSP72, HSP70, LC3A and LC3B in SOD1-G93A mice at several time points. Mice were stimulated for 1 hour, and then were sacrificed 24 hours or 48 hours later. Controls were C57 mice without the SOD1 gene expressed, and mice were 120 days of age. Data was normalized to GAPDH. Stimulation resulted in a large and sustained rise in HSP70 and a slower rise in HSP72. Following stimulation, LC3A and LC3B rise within 24 hours then decline.

### Example 11: Anodal msDCS Increases Survival of Spinal Motor Neurons

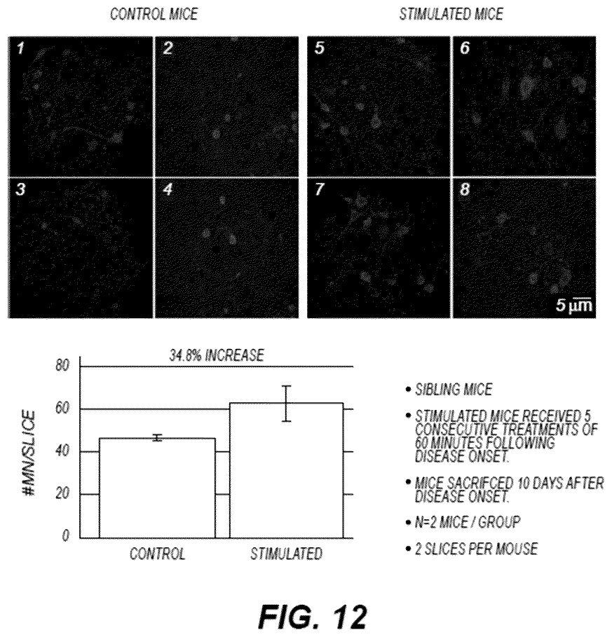

In ALS, motor neuron hyperexcitability results in eventual motor neuron death. We examined the effect of msDCS on motor neuron survival. FIG. 12 exhibits the effects of anodal msDCS on the survival of spinal motor neurons of SOD1-G93A mice. Sibling SD1-G93A mice were separated into stimulated and non-stimulated groups. Stimulated mice received 5 consecutive daily treatments of 60 minutes following disease onset. Mice were sacrificed 10 days after disease onset. Motor neurons were counted after 2 slices were extracted per mouse. Micrographs show 4 sections of the same slice for each example mouse. Stimulation resulted in the preservation of larger alpha motor neurons and an overall 34.8% increase in motor neuron count after stimulation as compared to non-stimulated carriers.

### Example 12: Anodal msDCS Increases Survival of SOD1-G93A Mice

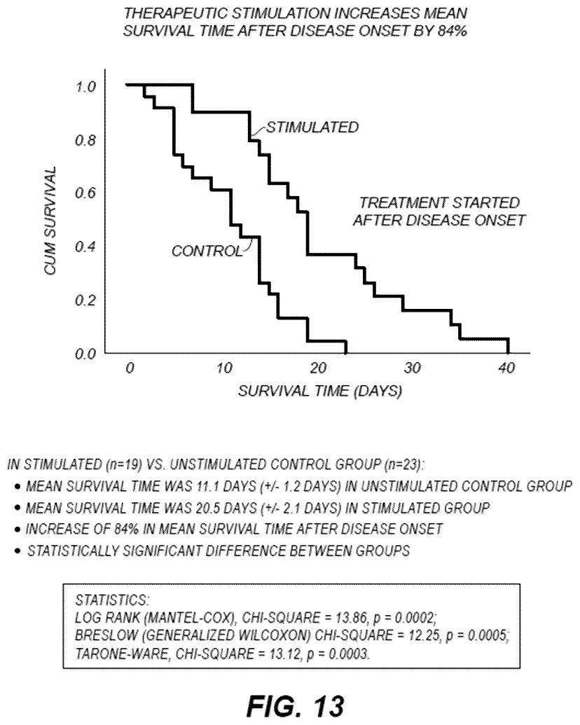

Our premise is that since msDCS can suppress hyperexcitable spinal motor neurons and, it can have effects beyond motor function. Using a therapeutic stimulation paradigm (5 days/week, 3.3 A/m² current density) in SOD1-G93A mice (starting at disease onset), we assessed survival in control vs. stimulated mice (gender balanced between groups). FIG. 13 is a Kaplan-Meier plot showing the effects of anodal msDCS on the survival of SOD1-G93A mice when treatment starts immediately after symptom onset. Symptom onset was defined as a score of 80 or less in the grid walking test. Stimulated carrier mice survived for 20.5 days (±2.1 days) on average while non-stimulated carrier mice survived for 11.1 days (±1.2 days) on average, leading to an 84% increase in mean survival time after disease onset for stimulated mice. N=19 stimulated mice; 23 non-stimulated mice. Log Rank (Mantel-Cox), Chi-Square=13.86, p=0.0002; Breslow (Generalized Wilcoxon) Chi-Square=12.25, p=0.0005; Tarone-Ware, Chi-Square=13.12, p=0.0003.

### Example 13: Anodal msDCS Improves Motor Function in TDP-43 Mice

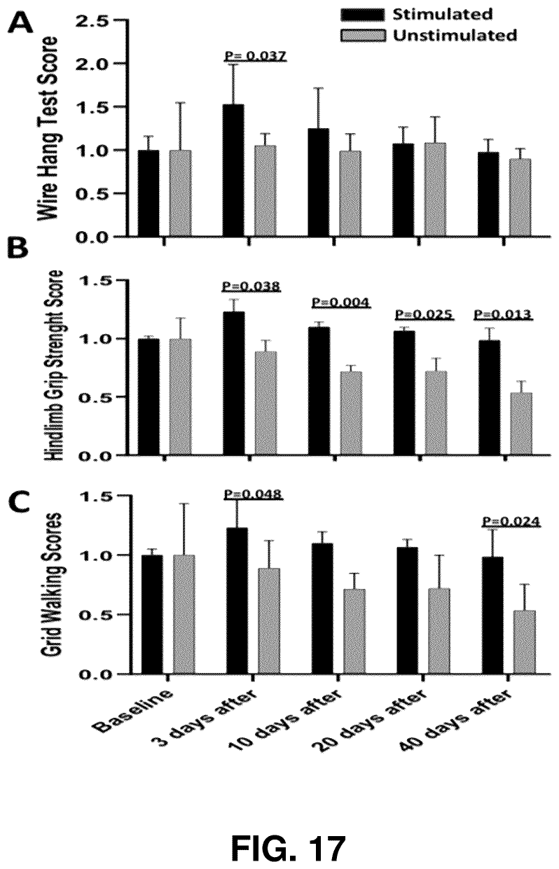

We established a colony of and began working with the TDP-43 mouse model of ALS (lines 23 and 60) to investigate the effects of msDCS on motor function and cytoplasmic TDP-43 aggregation, with our hypothesis being that stimulation will improve motor function and decrease TDP-43 aggregation as we saw in the SOD1-G93A model. TDP-43 mice were subjected to a comprehensive battery of motor function tests consisting of grip strength, grid walking and wire hang tests. They were then randomly divided into two groups, with one group receiving anodal msDCS 3×/week (1 hour/session) for 3 weeks (9 total treatments), while the other group served as a control. Following the last stimulation, testing was repeated and the results were striking. The stimulated group showed statistically significant improvements in all categories (grip, grid walking and wire hang) at 3-days post-stimulation compared to their results before the stimulations or their unstimulated littermates. This improvement, while declining over time, was still significant 40 days after treatment (FIG. 17). Mice were matched between groups: mice were taken from the same litter and pseudorandomized so that the same number of males and females were equally divided between the groups. Genotyping was done before group selection, and copy number was balanced. A315T mice were fed a soft gel diet after disease onset.

### Example 14: Anodal msDCS Reduces Cytosolic TDP-43 Protein Aggregates in Spinal Motor Neurons of TDP-43 Mice

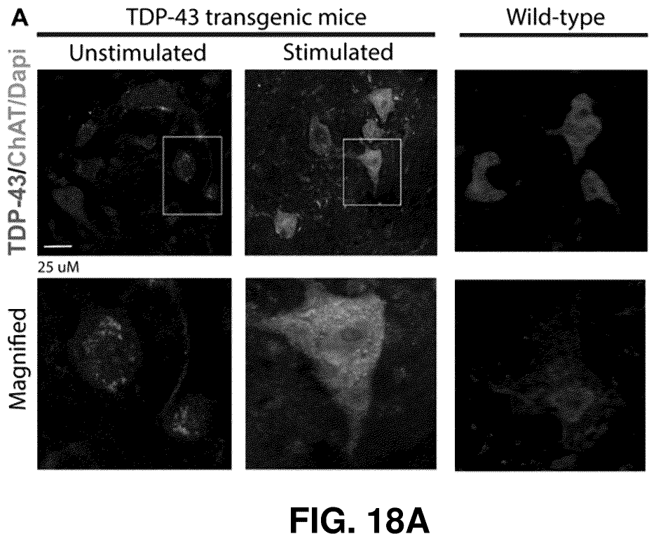

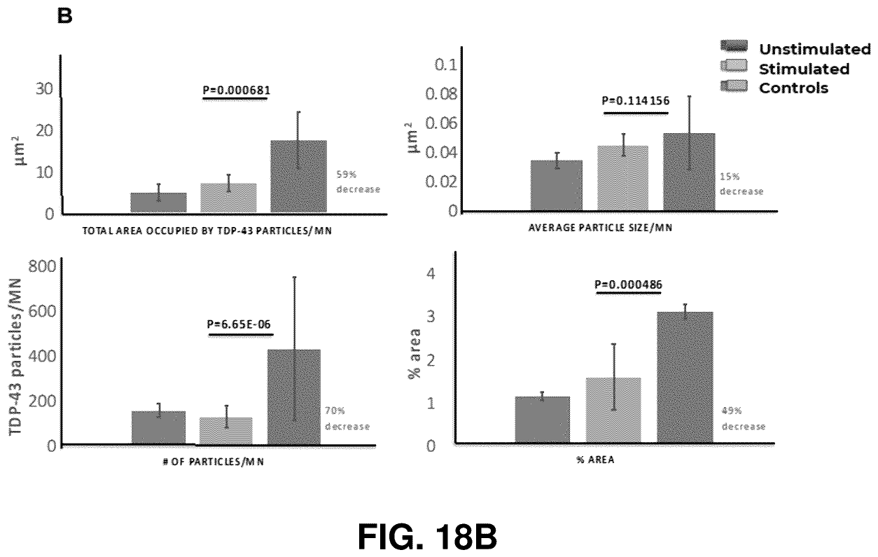

We examined TDP-43 protein levels in these same mice using immunohistochemistry. We analyzed the amount, size and area occupied by particles positive for TDP-43 in lumbar spinal motor neurons, with image analysis being blinded. In unstimulated TDP-43 mice (6 months age), TDP-43 was found atypically outside the nucleus, and stress granules were apparent in the cytoplasm. In stimulated TDP-43 mice, however, treatment decreased TDP-43 staining, with staining localized to the nucleus (FIG. 18A). Quantification of cytoplasmic TDP-43 protein aggregates is shown in FIG. 18B. A robust and statistically significant 70% reduction in number of particles/motor neuron was seen in TDP-43 mice treated with msDCS as compared to untreated control TDP-43 mice, as well as a reduction in total cytoplasmic area occupied by TDP-43 particles (also significant). This is the first time that any form of neuromodulation has been shown to reduce cytosolic TDP-43 protein aggregates in spinal motor neurons of an ALS model, and indicates msDCS could be therapeutically beneficial in a large majority of ALS cases.

### Example 15: Embodiment of Hyperexcitability Suppression Approach in Humans with ALS

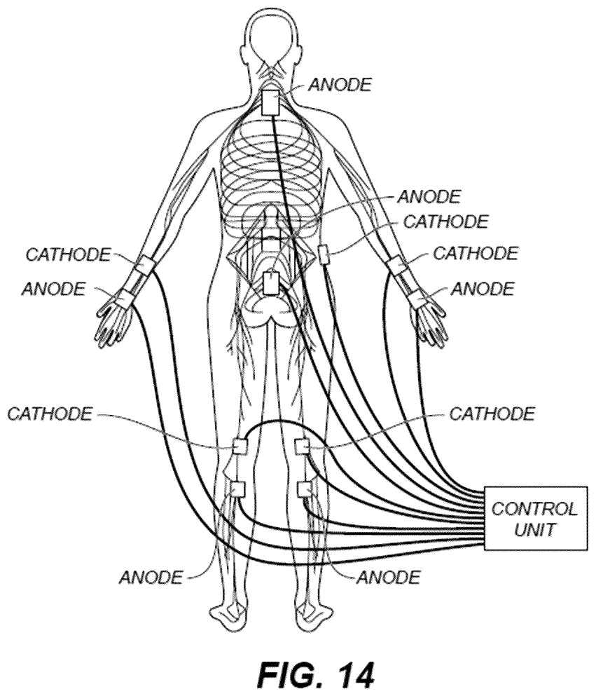

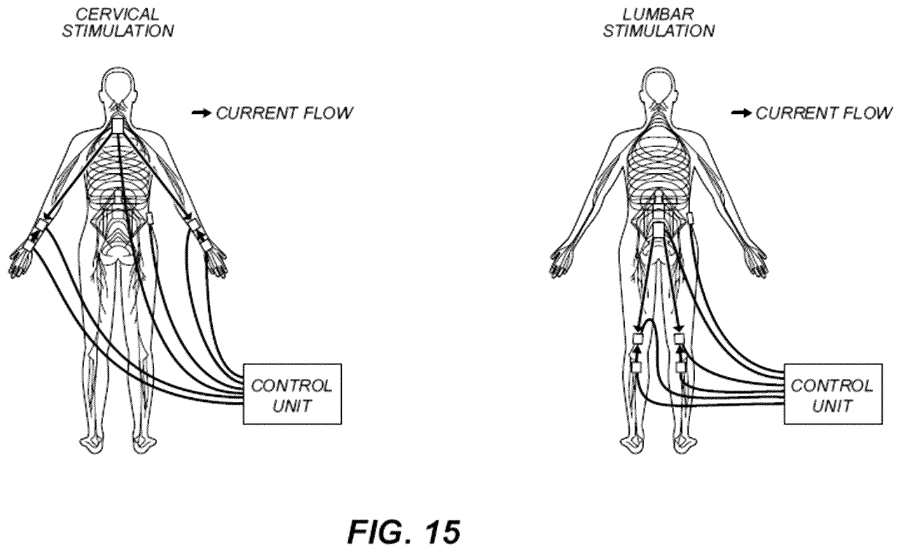

FIG. 14 illustrates an embodiment of a novel hyperexcitability suppression approach to treating ALS consisting of a control unit applying msDCS and skin-surface electrodes applied along the spinal column and the peripheral nerves of the four limbs. Anodes are positioned along the spinal column with a return electrode on the anterior iliac crest. Peripheral electrodes are positioned on each limb, with cathode positioned more proximally than anode. Current from the spinal anode flows both across the spinal cord (to return electrode) and down the limb (to peripheral cathodes). FIG. 15 shows an embodiment of a multi-channel unit that is used to sequentially treat ALS patients from the cervical to the lumbar spine to reduce spasticity and slow down disease progression. Each channel is limited to sourcing no more than 5 mA DC with a maximum current density of 0.56 mA/cm² for the smallest electrodes (below safety limits specified by applicable standards).

FIG. 16 illustrates an embodiment of using bi-hemispheric cortical stimulation to preserve cortical neurons using the same msDCS technology. Current flows from the cortical anodes to the spinal cathode. It will be appreciated that FIG. 16 shows forming an msDCS system with two current paths with two anodes and one cathode. It will be further understood that such embodiment enables simultaneous stimulation of a target from both circuits. However it will be further appreciated that either the left or right side circuit of FIG. 16 can be used alternatively in sequence, or just on its own, in practice of embodiments of the msDCS invention. All such configurations are encompassed within the practice of msDCS.

Aspects of the present invention, in stimulation systems, devices and practices, variously apply anodal msDCS to (a) reduce neuronal spinal excitability long-term, (b) slow the progression of muscle weakness, and (c) significantly increase lifespan of stimulated SOD1-G93A mice by 84%. Additionally, practice of our anodal msDCS variously: (a) reduces the expression of mutant SOD1 protein, (b) reduces the expression of and/or cytosolic protein aggregation of TDP-43, (c) reduces expression of elevated NKCC1, (d) reduces expression of elevated tau, (e) increases expression of HSP70 and HSP72, and (f) increases expression of LC3A and LC3B. Furthermore, aspects of our anodal msDCS technology can enhance clearance of misfolded proteins by modulating the proteolytic systems of autophagy and the proteasome. The present invention provides systems and devices for reducing spinal excitability and clearing toxic proteins from neurons in the spinal cord and elsewhere, and can provide disease-modifying stimulation for intervention against ALS and other neurodegenerative diseases.

While these teachings have been described in terms of specific embodiments, it will be evident in view of the foregoing description that numerous alternatives, modifications and variations will become apparent to those skilled in the art. Accordingly, these teachings are intended to encompass all such alternatives, modifications and variations which fall within the scope and spirit of the present teachings, and according to such claims as they are allowed after successful prosecution of this and other non-provisional applications based hereon in whole or in part.

What is claimed is:

1. A stimulation system for treating motor neuron and neurodegenerative diseases by regulating protein degradation pathways and autophagy associated with any of the spinal cord, the brain and/or a peripheral nerve, comprising:
a direct current voltage source having a plurality of terminals;
a first of the terminals for connecting a first electrode to the direct current voltage source; the first electrode for stimulating at or proximate to a target, said target including any one of a dorsal aspect of a spinal cord, a cranium, and a peripheral nerve of a vertebrate being;
a second of the terminals for connecting a second electrode to the direct current voltage source; the second electrode for placement at a position remote from the first electrode; the first and second electrodes being oppositely charged and configured to form a current path for stimulating said target; and
a controller component configured to control said current flow between said electrodes across said current path flow for stimulation of said target, said controller configure to deliver said current flow for a predetermined time period and repeat stimulation for a predetermined number of times; said stimulation promoting increased biological activity of, gene expression of, or protein expression of any member of the set of the following biological macromolecules: HSP70, HSP72, LC3B and LC3A.

2. The stimulation device of claim 1, wherein said controller component is further configured to provide said direct current flow at an intensity and polarity to change biological activity of, or level of gene expression of, or protein expression of, or cytoplasmic protein aggregation of the gene products of ALS-associated genes, including any of FUS (FUS protein), TARDBP (TDP-43 protein), and C9orf72, (C9orf72 protein), at at least one of said spinal cord, brain, and peripheral nerve.

3. The stimulation device of claim 1, wherein said motor neuron disease is a disease selected from the group consisting of amyotrophic lateral sclerosis, primary lateral sclerosis, progressive muscular atrophy, progressive bulbar palsy, spinal muscular atrophy and post-polio syndrome.

4. The stimulation device of claim 1, wherein the controller component is configured to provide said current flow including at least one of constant, continuous, pulsed, intermittent, varying, and non-varying current flow.

5. The stimulation device of claim 1, wherein at least one of the first and second electrodes is implanted.

6. The stimulation device of claim 1, wherein at least one of the controller component and the direct current voltage source are disposed in a wearable housing.

7. The stimulation device of claim 1, further including a plurality of said electrodes for forming a plurality of pairs of said electrodes; a second pair of said terminals for forming a second pair of electrodes, said second pair of electrodes connecting to said direct current voltage source; the first pair of electrodes for stimulation of a first location on said being and said second pair of electrodes for stimulation of a second location on said being, said locations selected as at or proximate to said dorsal aspect of said spinal cord, and on said cranium, and at said peripheral nerve, to deliver multi-site stimulations simultaneously to at least one of said spinal cord, brain, and peripheral nerve.

8. The stimulation device of claim 7, wherein said biological activity of or said level of gene expression or protein expression or cytoplasmic protein aggregation of any of NKCC1, SOD1, TDP-43 and tau is decreased.

9. The stimulation device of claim 7, wherein said biological activity of or said level of gene expression and/or protein expression of any of HSP70, HSP72, LC3A and LC3B is increased.

10. The system of claim 1, wherein said controller is further configured to deliver said current flow for a predetermined time period and repeat stimulation for a predetermined number of times for treatment of one of the set including classic amyotrophic lateral sclerosis, familial SOD1 ALS, progressive muscular atrophy (PMA), frontotemporal lobar degeneration (FTLD), ALS-FTLD, Alzheimer's disease (AD), dementia with Lewy bodies, primary lateral sclerosis (PLS), Parkinson's disease, Huntington's disease, hippocampal sclerosis, limbic-predominant age-related TDP-43 encephalopathy (LATE)/cerebral age-related TDP-43 with sclerosis (CARTS), chronic traumatic encephalopathy (CTE), Perry disease, and multisystem proteinopathy.

11. A system for treatment of amyotrophic lateral sclerosis (ALS) and other neurodegenerative diseases linked to cytoplasmic aggregation of TDP-43 proteins in neurons of vertebrate beings, to reduce degenerative processes in said neurons, the system comprising:
a controller component configured to regulate delivery of direct current stimulation to a vertebrate being having, or being at risk for, cytoplasmic aggregation of TDP-43 proteins in neurons of said being,
a first stimulation component configured to deliver said direct current stimulation to a target involved neural region at at least one of the spinal cord and the brain of said being;
a stimulation component defining a therapy circuit, said circuit having an active stimulation pole and a reference pole, said therapy circuit configured to provide said direct current stimulation between said active stimulation pole and said reference pole across said target region to reduce cytoplasmic aggregation of TDP-43 proteins in involved neurons, and
said controller configured for regulating said delivery of direct current stimulation for treatment of said involved neurons of said involved neural region for a predetermined time period and at a predetermined current intensity, to decrease said cytoplasmic aggregation of TDP-43 protein in said involved neurons.

12. A method of treating motor neuron disease selected from the group including amyotrophic lateral sclerosis, primary lateral sclerosis, progressive muscular atrophy, progressive bulbar palsy, spinal muscular atrophy and post-polio syndrome, in a vertebrate being, comprising the steps of:
defining an anodal direct current flow path between an A electrode and a B electrode pair of a direct current source, wherein the direct current is at least one of constant, continuous, pulsed, intermittent, varying and non-varying,
applying the direct current flow for stimulation between said A electrode and B electrode pair with the A and B electrodes oppositely charged, and the A electrode as anode and being at a position at or proximate to a first location of a dorsal aspect of a spinal cord of a vertebrate being or with the A electrode being at a first location on a cranium of a vertebrate being; and
placing the B electrode as cathode at a second position remote from the A electrode position,
applying said anodal direct current flow at an intensity and for a period of time to modulate biological activity of or level of gene expression or protein expression levels of or cytoplasmic protein aggregation of a target protein associated with a motor neuron disease, and
providing said anodal direct current flow to effect at least one of i) a decrease in the biological activity of, or level of gene expression of, or level of protein expression of, or cytoplasmic protein expression of, at least one of the following biological macromolecules: NKCC1, SOD1, TDP-43 and tau, and ii) an increase in biological activity of, or level of gene expression or level of protein expression of, at least one of the following biological macromolecules: HSP70, HSP72, LC3A and LC3B, in said being.

13. The method of claim 12, further comprising the steps of:
moving said A electrode to a second location of a dorsal aspect of a spinal cord of a vertebrate being or to a second location on a cranium of a vertebrate being;
placing said B electrode at a second position remote from the A electrode; and
applying the direct current stimulation at an intensity and for a period of time sufficient to modulate biological activity of or level of gene expression and/or protein expression levels of a target protein associated with ALS.

14. The method of claim 13, further comprising repeating the treatment one or more times.

15. The method of claim 13, wherein a plurality of A electrodes are located at or proximate to a plurality of positions along the dorsal aspect of the spinal cord and a plurality of B electrodes are placed at positions remote from the plurality of A electrodes.

16. The method of claim 15 wherein a plurality of A electrodes are located at a plurality of positions on the cranium associated with movement control and a plurality of B electrodes are placed at positions remote from the plurality of A electrodes.

17. The method of claim 16, wherein said stimulation applied between the plurality of A and B electrodes is applied simultaneously or sequentially.

18. A method of treating ALS in a vertebrate being, comprising the steps of:
applying a first stimulation between an A electrode and a B electrode of a direct current source with the A electrode being at or proximate to a first location of a dorsal aspect of a spinal cord of a vertebrate being;
applying a second stimulation between a C electrode and a D electrode of a direct current source with the C electrode being at a first location on a cranium of a vertebrate being; and
applying the direct current stimulation at an intensity and for a period of time sufficient to modulate biological activity of or level of gene expression or protein expression levels or cytoplasmic protein aggregation of a target protein associated with ALS;
placing the B electrode at a position remote from the A electrode and the A and B electrodes are oppositely charged, and the D electrode is placed at a position remote from the C electrode and the C and D electrodes are oppositely charged,
wherein the direct current is at least one of constant, continuous, pulsed, intermittent, varying and non-varying;
provide said direct current flow at an intensity, polarity and for a period of time to effect at least one of i) a decrease in biological activity of, or level of gene expression, or protein expression, or cytoplasmic protein expression of, at least one of the following biological macromolecules: NKCC1, SOD1, TDP-43 and tau, and ii) an increase in biological activity of, or level of gene expression or protein expression of, at least one of the following biological macromolecules: HSP70, HSP72, LC3A and LC3B, in said being.

19. The method of claim 18, wherein the first and second stimulations are applied simultaneously or sequentially.

20. The method of claim 18, further comprising the steps of:
moving said A electrode to a second location of a dorsal aspect of a spinal cord of a vertebrate being;
placing said B electrode at a second position remote from the A electrode; and
applying the direct current stimulation at an intensity and for a period of time sufficient to modulate biological activity of or level of gene expression or protein expression levels of or cytoplasmic protein aggregation of a target protein associated with ALS.

21. The method of claim 18, further comprising the steps of:
moving said C electrode to a second location on a cranium of a vertebrate being;
placing said D electrode at a second position remote from the C electrode; and
applying the direct current stimulation at an intensity and for a period of time sufficient to modulate biological activity of or level of gene expression and/or protein expression levels of or cytoplasmic protein aggregation of a target protein associated with ALS.

22. The method of claim 21, further comprising repeating the steps one or more times.

23. The method of claim 21, wherein a plurality of A electrodes are located at or proximate to a plurality of positions along the dorsal aspect of the spinal cord and a plurality of B electrodes are placed at positions remote from the plurality of A electrodes.

24. The method of claim 21, wherein a plurality of C electrodes are located at a plurality of positions on the cranium associated with movement control and a plurality of D electrodes are placed at positions remote from the plurality of C electrodes.

25. The method of claim 23, wherein said stimulation applied between the plurality of A and B electrodes and between the plurality of C and D electrodes is applied simultaneously or sequentially.

26. The method of claim 18, further comprising the step of:
applying a third stimulation between an E electrode and an F electrode of a direct current source with the E electrode being at or proximate to a first location of a first peripheral nerve of a vertebrate being; and
wherein the F electrode is placed at a position remote from the E electrode and the E and F electrodes are oppositely charged.

27. The method of claim 26, wherein the first, second and third stimulations are applied simultaneously or sequentially.

28. The method of claim 26, further comprising the steps of:
moving said E electrode to a second location at or proximate to the first peripheral nerve or to a location at or proximate to a second peripheral nerve;
optionally placing said F electrode at a second position remote from the E electrode; and applying the direct current stimulation at an intensity and for a period of time sufficient to modulate biological activity of or level of gene expression or protein expression levels of or cytoplasmic protein aggregation of a target protein associated with ALS.

29. The method of claim 28, further comprising repeating the steps one or more times.

30. The method of claim 26, wherein a plurality of E electrodes are located at a plurality of positions along the peripheral nerve or are located at or proximate to a plurality of peripheral nerves and a plurality of F electrodes are placed at positions remote from the plurality of E electrodes.

31. The method of claim 30, wherein said stimulation applied between the plurality of E and F electrodes is applied simultaneously or sequentially.

32. The method of claim 26, wherein said peripheral nerve innervates a skeletal muscle.

33. The method of claim 26, wherein said period of time comprises a series of stimulation sessions on 1 or more days, the days being consecutive or non-consecutive.

34. The method of claim 18, wherein said biological activity of or said level of gene expression and/or protein expression of NKCC1, SOD1, TDP-43 or tau is decreased.

35. The method of claim 18, wherein said biological activity of or said level of gene expression and/or protein expression of HSP70, HSP72, LC3A or LC3B is increased.
